(19) United States
(12) Patent Application Publication
(10) Pub. No.: US 2026/0130856 A1
(43) Pub. Date: May 14, 2026

(54) LIPOSOME COMPOSITIONS FOR DELIVERY OF COMPOUNDS AND METHODS THEREOF

(71) Applicant: NanoTech Pharma Inc., Hillsborough, NJ (US)

(72) Inventors: Xian Xu, Monmouth Junction, NJ (US); Fang Liu, Flemington, NJ (US)

(21) Appl. No.: 19/121,113

(22) PCT Filed: Oct. 13, 2023
(86) PCT No.: PCT/US23/76882

§ 371 Date: Apr. 14, 2025

Related U.S. Application Data
(60) Provisional application No. 63/416,483, filed on Oct. 14, 2022.

(51) Int. Cl. A61K 9/1271; A61K 31/4439; A61K 31/444; A61K 31/4709; A61K 31/4985; A61K 31/506; A61K 31/517; A61K 31/53; A61K 31/58; A61K 31/704; A61K 38/07; A61K 47/40; A61P 35/00

(52) U.S. Cl. CPC A61K 9/1271; A61K 31/4439; A61K 31/444; A61K 31/4709; A61K 31/4985; A61K 31/506; A61K 31/517; A61K 31/53; A61K 31/58; A61K 31/704; A61K 38/07; A61K 47/40; A61P 35/00

(57) ABSTRACT

The present disclosure provides liposome particles containing one or more active pharmaceutical ingredients (APIs), including at least one poorly water-soluble API, within the interior aqueous compartment of the particles, pharmaceutical compositions thereof, and use of the drug-loaded liposomes and pharmaceutical compositions thereof for treatment of patients, including various types of cancer patients, to achieve synergistic therapeutic effects. Treatment kits, dosage forms, and methods of making the drug-loaded liposomes and pharmaceutical compositions thereof are also disclosed.

### CROSS-REFERENCE TO RELATED APPLICATIONS

This application claims priority under 35 U.S.C. § 119(e) to U.S. Provisional Patent Application No. 63/416,483, filed on Oct. 14, 2022, the disclosure of which is incorporated herein by reference in its entirety.

### FIELD OF THE DISCLOSURE

The present disclosure relates to a liposome-based drug delivery system and methods thereof especially useful for delivery of poorly water-soluble drug molecules, for example, various anticancer agents.

### BACKGROUND OF THE DISCLOSURE

Among the nanoparticle-based drug delivery systems, liposome is a widely used pharmaceutical carrier with many unique characteristics, including the following: (1) prolonged drug circulation half-life mediated by the carrier; (2) reduced non-specific tissue uptake: (3) increased accumulation at the solid tumor or inflammation site through the enhanced permeation and retention (EPR) effect; (4) improved delivery specificity by surface modification with targeting agents for active targeting; (5) predominantly endocytosis uptake with the potential to bypass mechanisms of multidrug resistance; (6) a single delivery system carrying multiple drugs in the same vehicle which can lead to synchronized and controlled pharmacokinetics of each drug, resulting in improved efficacy through drug synergy; (7) ability to tailor the relative ratios of each compound based on its pharmacological disposition; (8) improved drug solubility and bioavailability; and (9) a sustained drug release profile (Mamot, C., et al., Drug Resist. Updates 2003, 6: 271-279).

To achieve desired high drug-to-lipid mass ratio and high drug encapsulation efficiency, remote loading or active loading has been developed to encapsulate drug compounds within the liposome. To perform drug remote loading, a transmembrane ion gradient is first generated by encapsulating trapping agents within the aqueous core of the liposome. During the remote loading process, amphipathic drug diffuses through the bilayer lipid membrane into the aqueous intravesicular space. Once inside the liposome, the drug interacts with pre-loaded trapping agents forming complexes in terms of precipitation, aggregation, or gelation. preventing membrane re-permeation, and therefore resulting in the accumulation of the drug within the vesicle (Hood, R., et al., Lab Chip, 2014, 14:3359-67).

Although remote loading can be used to efficiently load small molecule drugs, the payload has to have sufficient water solubility in order to obtain desired loading outcome. Remote loading of poorly water-soluble compounds is a long-standing technical challenge in the field. Hayes et. al. explored the use of aprotic solvents, e.g., dimethyl sulfoxide (DMSO) as solubility enhancer to increase the aqueous solubility of poorly water-soluble compounds for remote loading (Hayes, M. et. al., U.S. Ser. No. 10/004,759B2). However, only slight increase on drug encapsulation efficiency can be obtained by this approach. In addition, aprotic solvent such as DMSO can cause toxicity issues and also can adversely affect the physical stability of the liposome. Therefore, to date, it is still very challenging to efficiently load poorly water-soluble compound into liposomes via remote loading process with high drug-to-lipid mass ratio and high encapsulation efficiency.

### SUMMARY OF THE DISCLOSURE

The present disclosure provides liposome compositions encapsulating one, two or more drug compounds within the liposome aqueous core, and at least one of such compounds is a poorly water-soluble drug.

Also, the present disclosure provides a method to load poorly water-soluble compound into the liposome with high drug-to-lipid mass ratio and high drug encapsulation efficiency. Generally, the method involves the following: Firstly, using solubility improving agent(s) to increase the solubility and the concentration of the poorly water-soluble compound in the external (i.e., extraliposomal) aqueous medium, followed by using a remote drug loading approach to efficiently encapsulate the compound within the aqueous core of the liposome.

For the remote loading of poorly water-soluble weak amphipathic base, a pH gradient is established across the lipid membrane. It was surprisingly observed that the pH value in the liposome external medium is required to be lower than that of the internal medium. This observation is completely opposite to the conventional pH gradient approach used for the remote loading of weak bases reported previously (Madden, D., Chemistry and Physics of Lipids, 1990, 53:37-46), wherein the pH value in the external medium is higher than that of the internal medium.

In one aspect, the present disclosure provides drug-loaded liposome particles comprising an interior core and an exterior lipid bilayer membrane, wherein the lipid bilayer membrane comprises an inner layer having an inner surface enclosing the interior core and an outer layer forming an outer surface of the liposome particle; and the interior core comprises an aqueous liquid medium and one or more active pharmaceutical ingredients encapsulated by the bilayer membrane, wherein at least one of the active pharmaceutical ingredients is poorly water-soluble.

In another aspect, the present disclosure provides a pharmaceutical composition, comprising the drug-loaded liposome particles according to any embodiments disclosed herein and a liposome dispersion liquid medium.

In another aspect, the present disclosure provides a method of treating a subject in need of treatment with a therapeutic agent, the method comprising administering the subject a therapeutically effective amount of the drug-loaded liposome particles according to any embodiment disclosed herein or a pharmaceutical composition thereof.

In another aspect, the present disclosure provides a method of preparing liposomes loaded with one or more active pharmaceutical ingredients, including at least one poorly-water soluble active pharmaceutical ingredient.

In another aspect, the present disclosure provides a treatment kit comprising the drug-loaded liposome particles according to any embodiment disclosed herein or a pharmaceutical composition comprising the drug-loaded liposome particles according to any embodiment disclosed herein.

In an exemplary embodiment for the active loading of poorly water-soluble weak amphipathic base, the pH value of the external medium is 8.0 or 7.0 or 6.0 or 5.0 or 4.0 or 3.0 or 2.0 or 1.0 or 0.5 or 0.25 or 0.1 unit lower than that of the internal medium. In one embodiment, the internal medium pH is between 5.0 to 10.0 and the external medium pH is between 2.0 to 5.0.

In an exemplary embodiment, the disclosure provides a pharmaceutical formulation comprising a liposome having a membrane encapsulating an aqueous compartment. Encapsulated within the aqueous compartment are remote loading trapping agent(s) and one, two or more drug compounds, and at least one of such compounds is poorly water-soluble. In various embodiments, about 60%, about 70%, about 90%, about 95% or about 99% of each compound is encapsulated within the aqueous compartment of the liposome.

In another exemplary embodiment, liposomal compositions containing two or more therapeutic agents provided herein include liposomes stably associated therewith those compounds, and those encapsulated compounds have a drug-to-drug molar ratio that exhibits a non-antagonistic effect to relevant cells or tumor homogenates.

In one exemplary embodiment, the drug loaded liposomes can be prepared according to the following steps:
a) forming a lipid dispersion in a solution comprising trapping agent(s) and optionally buffering agent(s);
b) reducing liposome particle size at an elevated temperature;
c) substantially removing the trapping agent outside of the liposome thereby obtaining unloaded liposome;
d) separately, dissolving active pharmaceutical ingredient(s) in the presence of solubility improving agent(s) in aqueous solution. When two or more drugs are loaded, at least one of the drug compounds is poorly water soluble;
e) incubating said unloaded liposome with said drug or drug combination solution comprising solubility improving agent(s) at an elevated temperature thereby forming the drug loaded liposomes through active loading;
f) optionally, removing unloaded drug(s) and the solubility improving agent(s) outside the liposomes; and
g) optionally, forming dry form of the liposome product by lyophilization.

The technology presented in the disclosure enlarges the range of functional drug compounds amenable to encapsulation in liposomes. Other aspects and advantages will be better understood in view of the drawings, detailed description, examples, and claims that follow.

### BRIEF DESCRIPTION OF THE DRAWINGS

FIG. 1 illustrates the particle size distribution of the drug loaded liposomes characterized by dynamic light scattering. TEA-SOS was used as the trapping agent and sodium salt of SBE-β-CD was used as the solubility improving agent for poorly water-soluble drugs. (A) Carfilzomib liposome (B) Ceritinib liposome (C) Dasatinib liposome (D) Afatinib/Dasatinib co-loaded liposome (E) Doxorubicin/Carfilzomib co-loaded liposome and (F) Dasatinib/Ceritinib co-loaded liposome.

FIGS. 2A, 2B and 2C illustrates the in vitro evaluation of combo drug combinations for synergy on different type of cancer cells. Representative plot of combination index (CI) value as a function of cell growth inhibition (indicated by Fa) at different drug-to-drug molar ratios, where CI values of <1, ˜1, and >1 indicate synergy, additivity, and antagonism, respectively. 5 FIG. 2A: Combination of afatinib (AFA) and dasatinib (DAS) on HCC-827 non-small cell lung cancer cell line. FIG. 2B: Combination of dasatinib (DAS) and ceritinib (CER) on HCC-827 non-small cell lung cancer cell line. FIG. 2C: Combination of carfilzomib (CAR) and doxorubicin (DOX) on H929 myeloma cell line.

FIGS. 3A and 3B illustrates the morphological characterization of the carfilzomib-loaded liposome by using cryogenic transmission electron microscopy (cryo-TEM). FIG. 3A: Low magnification image; FIG. 3B: High magnification image.

FIG. 4 illustrates the in vivo pharmacokinetics (PK) profile in BALB/c mice of both free carfilzomib solution and carfilzomib-loaded liposomes.

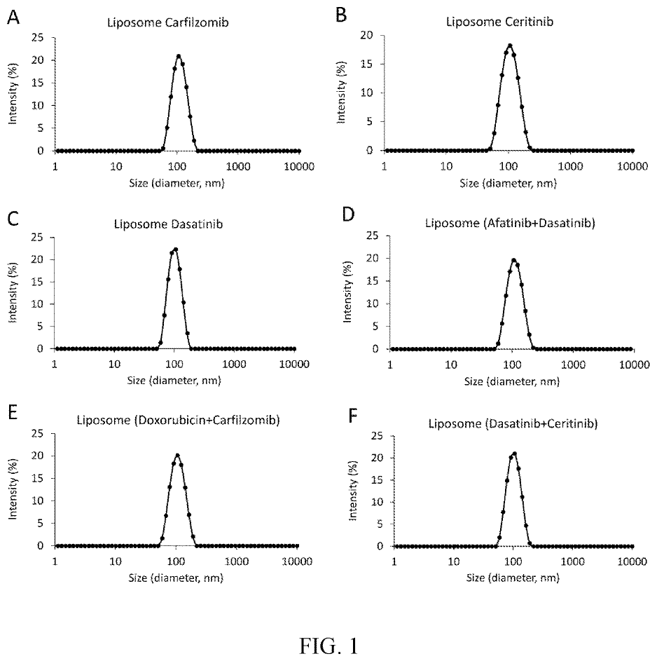

### DETAILED DESCRIPTION OF THE DISCLOSURE

When liposomes are used for the delivery of therapeutical compounds, it is generally desirable to load the liposomes with high concentration of drug molecules, resulting in a high drug-to-lipid mass ratio, since this reduces the amount of liposomes to be administered per treatment to obtain the required therapeutic effect of the compound. Also, since some of the lipids used for liposome preparation can generate undesired toxicity by themselves when the dose reaches a certain threshold level, thereby using a high drug-to-lipid mass ratio may avoid such adverse effects. In addition, it is also highly desirable to attain a high drug encapsulation efficiency for a liposome drug product, since it is well known that liposome formulation of a compound dramatically alters its pharmacokinetics profile, resulting in enhanced therapeutic efficacy, reduced drug-related toxicity effect, and overall improved therapeutic index.

Both the drug-to-lipid mass ratio and the drug encapsulation efficiency for poorly water-soluble compound in a liposome formulation can be increased firstly by using solubility improving agents to increase the concentration of the poorly water-soluble compound in the extraliposomal aqueous medium, followed by using a remote drug loading method to efficiently encapsulate the compound within the aqueous core of the liposome.

The present disclosure provides liposomes encapsulating one, two or more drug compounds within the liposomal aqueous core, and at least one of such compounds is a poorly water-soluble drug. The disclosure provides methods of making such liposomes, formulations containing such liposomes and methods of making liposome formulations of the disclosure.

In one aspect, the present disclosure provides drug-loaded liposome particles comprising an interior core and an exterior lipid bilayer membrane, wherein the lipid bilayer s membrane comprises an inner layer having an inner surface enclosing the interior core and an outer layer forming an outer surface of the liposome particle; and the interior core comprises an aqueous liquid medium and one or more active pharmaceutical ingredients encapsulated by the bilayer membrane, wherein at least one of the active pharmaceutical ingredients is poorly water-soluble.

In some embodiments, the drug-loaded liposome particles comprise an interior core and an exterior lipid bilayer membrane, wherein the lipid bilayer membrane comprises an inner layer having an inner surface enclosing the interior core and an outer layer forming an outer surface of the liposome particle; wherein the interior core comprises an aqueous liquid medium and one or more active pharmaceutical ingredients encapsulated by the bilayer membrane, wherein at least one of the active pharmaceutical ingredients is poorly water-soluble: wherein the aqueous liquid medium of the interior core comprises a trapping agent and optionally a buffering agent; and wherein the drug-loaded liposome particles have a mean particle size between 10 nm and 450 nm, optionally between 25 nm and 300 nm or between 50 nm and 200 nm.

In some embodiments, in the drug-loaded liposome particles, the lipid bilayer membrane comprises:
a) a phospholipid selected from phosphatidylcholine (e.g., HSPC, DSPC, DPPC and DMPC), phosphatidylglycerol (e.g., DSPG, DPPG and DMPG), phosphatidylinositol, glycerol glycolipids, sphingoglycolipids (e.g., sphingomyelin), and combinations thereof, wherein the phospholipid is in an amount of at least 10 mol % of the total lipid present in the liposome particle;
b) cholesterol, or a derivative thereof, in an amount of from 5 mol % to 50 mol % of the total lipid present in the liposome particle; and
c) a conjugated lipid, which inhibits aggregation of liposomes, in an amount of from 0 mol % to 10 mol %, sometimes preferably from 0.1 mol % to 10 mol %, sometimes preferably from 1 mol % to 10 mol %, sometimes preferably from 2 mol % to 8 mol %, sometimes preferably from 3 mol % to 6 mol %, of the total lipid present in the liposome particle.

In some embodiments, in the drug-loaded liposome particles, the conjugated lipid that inhibits aggregation of liposomes comprises a polyethyleneglycol (PEG)-lipid conjugate.

In some embodiments, in the drug-loaded liposome particles, the PEG has an average molecular weight in the range of about 1,500 Daltons to about 2,500 Daltons.

In some embodiments, in the drug-loaded liposome particles, the PEG has an average molecular weight of about 2,000 Daltons.

In some embodiments, in the drug-loaded liposome particles, the PEG-lipid conjugate is mPEG2000-DSPE or PEG2000-DMG.

In some embodiments, in the drug-loaded liposome particles, the liquid medium in the interior core comprises a trapping agent without a buffering agent.

In some embodiments, in the drug-loaded liposome particles, the liquid medium in the interior core comprises both a trapping agent and a buffering agent.

In some embodiments, in the drug-loaded liposome particles, the buffering agent is selected from acetic acid, citric acid, histidine, HEPES, lactic acid, succinic acid, phosphoric acid, tromethamine (Tris), and salts thereof.

In some embodiments, in the drug-loaded liposome particles, the trapping agent is selected from ammonium sulfate; ammonium or substituted ammonium salts of polyanionized sulfobutyl ether cyclodextrin; ammonium or substituted ammonium salts of polyanionized sulfated carbohydrates; ammonium or substituted ammonium salts of polyphosphate; metal salts; and combinations thereof.

In some embodiments, in the drug-loaded liposome particles, the ammonium salts of polyanionized sulfobutyl ether cyclodextrin are selected from TEA-SBE-α-cyclodextrin, TEA-SBE-β-cyclodextrin, TEA-SBE-γ-cyclodextrin. Tris-SBE-α-cyclodextrin, Tris-SBE-β-cyclodextrin and Tris-SBE-γ-cyclodextrin; the ammonium salts of polyanionized sulfated carbohydrates are selected from TEA-SOS and Tris-SOS; the ammonium salts of polyphosphate are selected from triethylammonium inositol hexaphosphate and tris(hydroxymethyl) aminomethane inositol hexaphosphate; and the metal salt is selected from calcium, copper, zinc, magnesium, manganese, nickel, or cobalt salts of acetate, carbonate, citrate, halide, sulfate, and gluconate.

In some embodiments, the drug-loaded liposome particles have a mean particle size between 10 nm and 450 nm, sometimes preferably between 25 nm and 300 nm, and sometimes preferably between 50 nm and 200 nm.

In some embodiments, in the drug-loaded liposome particles, the active pharmaceutical s ingredients are selected from afatinib, abemaciclib, abiraterone, acalabrutinib, alectinib, almonertinib, alpelisib, anlotinib, apatinib, avapritinib, axitinib, baricitinib, belinostat, binimetinib, bortezomib, bosutinib, brigatinib, bupivacaine, cabozantinib, capecitabine, carfilzomib, capmatinib, ceritinib, cobimetinib, copanslisib, crizotinib, dabrafenib, dacomitinib, dasatinib, delanzomib, docetaxel, doxorubicin, duvelisib, enasidenib, encorafenib, entrectinib, erdafitinib, erlotinib, everolimus, fedratinib, fostamatinib, fruquintinib, gefitinib, gemcitabine, gilteritinib, glasdegib, icotinib, ibrutinib, idarubicin, idelalisib, imatinib, ivosidenib, ixazomib, ixabepilone, lapatinib, larotrectinib, lenalidomide, lenvatinib, lorlatinib, marizomib, midostaurin, mitoxantrone, neratinib, netarsudil, nilotinib, nintedanib, niraparib, olaparib, oprozomib, osimertinib, paclitaxel, palbociclib, panobinostat, pazopanib, pemetrexed, pemigatinib, pexidartinib, ponatinib, pralsetinib, quizartinib, radotinib, regorafenib, ribociclib, ripretinib, rivastigmine, romidepsin, rucaparib, ruxolitinib, selpercatinib, selumetinib, sirolimus, sonidegib, sorafenib, sunitinib, talazoparib, tazemetostat, temsirolimus, tepotinib, tivozanib, tofacitinib, topotecan, trametinib, tucatinib, tucidinostat, upadacitinib, vandetanib, vemurafenib, venetoclax, vinorelbine, vismodegib, vorinostat, and zanubrutinib, and freebases, pharmaceutical salts, derivatives, and mixtures thereof.

In some embodiments, in the drug-loaded liposome particles, the active pharmaceutical ingredients are selected from:
a) Carfilzomib encapsulated alone;
b) Dasatinib encapsulated alone;
c) Ceritinib encapsulated alone;
d) Carfilzomib and doxorubicin co-encapsulated;
e) Dasatinib and ceritinib co-encapsulated;
f) Afatinib and dasatinib co-encapsulated;
g) Carfilzomib and doxorubicin in about 1:50 to about 1:1000 molar ratio;
h) Dasatinib and ceritinib in about 30:1 to about 1:30 molar ratio; and
i) Afatinib and dasatinib in about 30:1 to about 1:30 molar ratio.

In some embodiments, in the drug-loaded liposome particles, two or more, sometimes preferably two or three, and sometimes more preferably two, active pharmaceutical ingredients are co-encapsulated in the interior core. When two or more, such as two or three, active s pharmaceutical ingredients are encapsulated, at least one, sometimes preferably both or all three, pharmaceutical ingredients are poorly water soluble.

In another aspect, the present disclosure provides a pharmaceutical composition, comprising the drug-loaded liposome particles according to any embodiments disclosed herein and a liposome dispersion liquid medium.

In some embodiments, in the pharmaceutical composition, the liposome dispersion liquid medium comprises water, a buffering agent, and a tonicity modifier.

In some embodiments, in the pharmaceutical composition, two or more active pharmaceutical ingredients are co-encapsulated in the interior core of the liposome particles and can be released to function in a synergistic mode for efficacy.

In some embodiments, in the pharmaceutical composition, the synergistic mode comprises that the active pharmaceutical ingredients can maintain a synergistic molar ratio in blood for at least one hour after administration of the pharmaceutical composition to a subject.

In some embodiments, in the pharmaceutical composition, the synergistic molar ratio is a molar ratio such that when the ratio is provided to cancer cells relevant to the cancer in an in-vitro assay over a drug concentration range at which cell growth inhibition range is from about 0.20 to about 0.80 (i.e., the fraction of affected cells is in the range of about 20% to about 80%), a synergistic effect of at least 20% is exhibited within the cell growth inhibition range.

In some embodiments, in the pharmaceutical composition, two active pharmaceutical ingredients are encapsulated in the interior core of the liposome particles in a molar ratio in the range from about 1000:1 to about 1:1000, sometimes preferably from 500:1 to 1:500, sometimes more preferably from 100:1 to 1:100, sometimes more preferably from 50:1 to 1:50, and sometimes more preferably from 10:1 to 1:10, sometimes more preferably from 5:1 to 1:5, and sometimes more preferably from 2:1 to 1:2.

In some embodiments, in the pharmaceutical composition, the buffering agent in the liposome dispersion liquid medium is selected from acetic acid, citric acid, histidine, HEPES, lactic acid, succinic acid, phosphate salt, tromethamine (Tris), and salts thereof; and the tonicity modifier the liposome dispersion liquid medium is selected from sucrose, dextrose, mannitol, trehalose, and sodium chloride.

In some embodiments, in the pharmaceutical composition, the liposome dispersion liquid medium has a pH in the range of 5.0 to 10.0, sometimes preferably from 6.0 to 8.0, s sometimes more preferably 6.5 to 7.5, sometimes more preferably 6.8 to 7.2, and sometimes even more preferably 7.0.

In another aspect, the present disclosure provides a method of treating a subject in need of treatment with a therapeutic agent, the method comprising administering the subject a therapeutically effective amount of the drug-loaded liposome particles according to any embodiment disclosed herein or a pharmaceutical composition thereof.

In some embodiments, in the method of treatment, the subject is a cancer patient needing treatment by two or more cancer agents in a synergistic mode.

In some embodiments, the subject is a cancer patient needing treatment by two or more cancer agents in a synergistic mode; wherein the cancer is a cancer of the bladder (including accelerated and metastatic bladder cancer), breast (e.g., estrogen receptor positive breast cancer, estrogen, receptor negative breast cancer; HER-2 positive breast cancer; HER-2 negative breast cancer, progesterone receptor positive breast cancer, progesterone receptor negative breast cancer; estrogen receptor negative, HER-2 negative and progesterone receptor negative breast cancer (i.e., triple negative breast cancer); inflammatory breast cancer), colon (including colorectal cancer), kidney (e.g., transitional cell carcinoma), liver, lung (including small and non-small cell lung cancer, lung adenocarcinoma and squamous cell cancer), genitourinary tract. e.g., ovary (including fallopian tube and peritoneal cancers), cervix, prostate, testes, kidney, and ureter, lymphatic system, rectum, larynx, pancreas (including exocrine pancreatic carcinoma), esophagus, stomach, gall bladder, thyroid, skin (including squamous cell carcinoma), brain (including glioblastoma multiforme), head and neck (e.g., occult primary), and Soft tissue (e.g., Kaposi's sarcoma (e.g., AIDS related Kaposi's sarcoma), leiomyosarcoma, angiosarcoma, and histiocytoma).

In some embodiments, the cancer is selected from multiple myeloma, chronic myeloid leukemia, lung cancer including small and non-small cell lung cancers, lung adenocarcinoma and squamous cell cancer.

In another aspect, the present disclosure provides a method of preparing liposomes loaded with one or more active pharmaceutical ingredients, comprising the steps of.
a) preparing a lipid dispersion in a solution comprising a trapping agent(s) and optionally a buffering agent(s) to form a suspension comprising liposome particles;
b) reducing liposome particle size by heating the suspension to an elevated temperature (at or above 50° C.);
c) substantially removing the trapping agent in the suspension outside of the liposomes, thereby obtaining unloaded liposomes;
d) dissolving one or more active pharmaceutical ingredient(s) (API) in an aqueous solution in the presence of a solubility improving agent to obtain an API solution;
e) incubating the unloaded liposomes of step c) with the API solution of step d) comprising the solubility improving agent at an elevated temperature (at or above 50° C.), thereby forming liposome particles comprising an aqueous interior core loaded with the one or more API(s) encapsulated by a bilayer membrane of the lipid, wherein the drug-loaded liposome particles are suspended in an external liquid medium;
f) optionally further removing unloaded drug molecules and the solubility improving agent outside of the liposome particles obtained in step e) by dialysis, ultracentrifugation, or/and size exclusion chromatography; and
g) optionally forming dry liposome particulates loaded with the one or more API(s) by lyophilizing the liposome particles obtained in step e) or step f).

In some embodiments, in step a), the solution comprises a trapping agent without a buffering agent so that in the drug-loaded liposome particles prepared, the liquid medium in the interior core comprises a trapping agent without a buffering agent.

In some embodiments, in step a), the solution comprises both a trapping agent and a buffering agent so that in the drug-loaded liposome particles prepared, the liquid medium in the interior core comprises both the trapping agent and the buffering agent.

In some embodiments, in the method of preparing liposomes loaded with one or more active pharmaceutical ingredients, the solubility improving agent is selected from cyclodextrins and derivatives, polyvinylpyrrolidone, polyethylene glycol and derivatives, sorbitol, non-ionic surfactants, and combinations thereof.

In some embodiments, in the method of preparing liposomes loaded with one or more active pharmaceutical ingredients, the solubility improving agent is sulfobutylether-β-cyclodextrin or hvdroxypropyl-β-cyclodextrin, or a salt thereof.

In some embodiments, in the method of preparing liposomes loaded with one or more active pharmaceutical ingredients, the step e) of incubating the unloaded liposomes with the drug solution results in at least 50% of total API(s) being encapsulated within the aqueous interior core of the liposome particles and less than 50% of total API molecules existing in the external liquid medium.

In some embodiments, in the method of preparing liposomes loaded with one or more active pharmaceutical ingredients, at the beginning of drug loading step e), the liquid medium of the liposome interior core has a pH in the range from about 5.0 to about 10.0 and the exterior medium outside of the liposome particles has a pH in the range from about 2.0 to about 5.0.

In some embodiments, the process includes both step f) of removing unloaded drug molecules and the solubility improving agent outside of the liposome particles obtained in step e) by dialysis, ultracentrifugation, or/and size exclusion chromatography, and step g) of forming dry liposome particulates loaded with the one or more API(s) by lyophilizing the liposome particles obtained in step e) or step f). Such a process produces “dry” liposome particles loaded with the APIs.

In some embodiments, the process includes only step f) of removing unloaded drug molecules and the solubility improving agent outside of the liposome particles obtained in step e) by dialysis, ultracentrifugation, or/and size exclusion chromatography, without step g) of forming dry liposome particulates loaded with the one or more API(s) by lyophilizing the liposome particles obtained in step e) or step f). Such a process produces “wet” liposome particles loaded with the APIs.

In another aspect, the present disclosure provides a treatment kit comprising a first container comprising a plurality of the drug-loaded liposome particles according to any embodiment disclosed herein, and a second container comprising a liposome dispersion liquid medium, wherein the drug-loaded liposome particles and the liposome dispersion liquid medium can be mixed in either the first container or the second container to form a dispersion that is ready for administration to a subject in need of treatment; or, alternatively, comprising a container comprising a liposome pharmaceutical composition according to any embodiment disclosed herein ready for administration to a subject in need of treatment.

In some embodiments, sometimes preferred, the treatment kit comprises a first container comprising a plurality of the drug-loaded liposome particles according to any embodiment disclosed herein, and a second container comprising a liposome dispersion liquid medium, wherein the drug-loaded liposome particles and the liposome dispersion liquid medium can be mixed in either the first container or the second container to form a liposome dispersion that is ready for administration to a subject in need of treatment.

In some embodiments, sometimes preferred, the treatment kit comprises a single container comprising liposome pharmaceutical composition according to any embodiment disclosed herein ready for administration to a subject in need of treatment. The liquid pharmaceutical composition may be prepared directly from the liposome drug-loading process according to any embodiment disclosed herein or alternatively prepared from mixing isolated drug-loaded liposome particles, either wet or dried, with a liposome dispersion liquid medium.

In some embodiments, in the treatment kit, the liposome dispersion liquid medium comprises water, a buffering agent, and a tonicity modifier.

In some embodiments, in the treatment kit, the buffering agent the liposome dispersion liquid medium is selected from acetic acid, citric acid, histidine, HEPES, lactic acid, succinic acid, phosphate salt, tromethamine (Tris), and salts thereof; and the tonicity modifier is selected from sucrose, dextrose, mannitol, trehalose, and sodium chloride.

In some embodiments, in the treatment kit, the liposome dispersion liquid medium has a pH in the range of 5.0 to 10.0, preferably from 6.0 to 8.0.

In some embodiments, the treatment kit further comprises a syringe and/or needle suitable for administration of the liposome dispersion to a subject.

In some embodiments, the treatment kit further comprises an instruction on mixing the drug-loaded liposome particles with the liposome dispersion liquid medium for administration of the mixture to a subject in need of treatment.

As would be understood by a person of skill in the pertinent art, the present disclosure encompasses any and all reasonable combinations of any two or more embodiments described within each aspect of the disclosure.

In an exemplary embodiment, the disclosure provides a pharmaceutical formulation comprising a liposome having a membrane encapsulating an aqueous compartment. Encapsulated within the aqueous compartment are a remote loading trapping agent, optionally a buffering agent and one, two or more drug compounds, and at least one of such compounds is poorly water-soluble. In various embodiments, about 50%, about 70%, about 90%, about 95% or about 99% of each compound is encapsulated within the aqueous compartment of the liposome.

In an exemplary embodiment, the disclosure provides a pharmaceutical composition wherein the drug loading content is about 5 wt %, about 10 wt %, about 15 wt %, about 20 wt %, about 25 wt %, about 30 wt %, about 35 wt %, about 40 wt %, about 45 wt %, about 50 wt % or higher. The drug loading content in the final drug product is defined as follows:

$$Drug loading content ( wt \% ) = \frac{Total entrapped drug concentration}{Total entrapped drug concentration + Total lipid concentration} \times 100 \%$$

In an exemplary embodiment, the interaction of the poorly water-soluble compound with the solubility improving agent is by physical interaction (such as charge, hydrophobic interaction, or hydrogen binding) and the compound is not covalently attached to the solubility improving agent or to a component of the liposome.

Preferably, liposomal compositions containing two or more therapeutic agents provided herein will include liposomes stably associated therewith those compounds, and those encapsulated compounds have a drug-to-drug molar ratio that exhibits a non-antagonistic therapeutic effect to relevant cells or tumor homogenates.

The lipid-based delivery vehicles of the present disclosure may be used not only in parenteral administration but also in topical, nasal, subcutaneous, intraperitoneal, intramuscular, aerosol or oral delivery by the application of the delivery vehicle onto or into a natural or synthetic implantable device at or near the target site for therapeutic purposes or medical imaging and the like. Preferably, the lipid-based delivery vehicles of the disclosure are used in parenteral administration, most preferably, intravenous administration.

The preferred embodiments herein described are not intended to be exhaustive or to limit the scope of the invention to the precise forms disclosed. They are chosen and described to best explain the principles of the invention and its application and practical use to allow others skilled in the art to comprehend its teachings.

### I. Poorly Water-Soluble Compounds

As indicated above, the present disclosure provides liposomes encapsulating one, two or more therapeutic agents, and at least one of such compounds is poorly water-soluble. In the context of the present disclosure, the term “poorly water-soluble” means being insoluble or having a very limited solubility in water, more in particular having an aqueous solubility of less than or equal to 1 mg/mL, sometimes preferably 0.5 mg/mL, and sometimes more preferably 0.2 mg/mL. As used herein, water solubilities refer to compound solubility measured at ambient temperature, which is typically about 20-25° C. at a neutral pH. If solubility varies with the temperature in the range of 20-25° C., when the solubility is not higher than 1 mg/mL, sometimes preferably 0.5 mg/mL, and sometimes more preferably 0.2 mg/mL, at any point of the temperature range, it should be considered “poorly water-soluble.” In an exemplary embodiment, the water solubility of the compound is measured at a neutral condition, about pH 7.0, sometimes preferably between pH 6.9 and pH 7.1, sometimes more preferably between pH 6.8 and pH 7.2, and sometimes more preferably pH 6.5 and pH 7.5.

The term “active pharmaceutical ingredients” refers to the active components in a pharmaceutical drug product that produce the required effect on the body to treat a condition. Exemplary active pharmaceutical ingredients include the following: afatinib, abemaciclib, abiraterone, acalabrutinib, alectinib, almonertinib, alpelisib, anlotinib, apatinib, avapritinib, axitinib, baricitinib, belinostat, binimetinib, bortezomib, bosutinib, brigatinib, bupivacaine, cabozantinib, capecitabine, carfilzomib, capmatinib, ceritinib, cobimetinib, copanslisib, crizotinib, dabrafenib, dacomitinib, dasatinib, delanzomib, docetaxel, doxorubicin, duvelisib, enasidenib, encorafenib, entrectinib, erdafitinib, erlotinib, everolimus, fedratinib, fostamatinib, fruquintinib, gefitinib, gemcitabine, gilteritinib, glasdegib, icotinib, ibrutinib, idarubicin, idelalisib, imatinib, ivosidenib, ixazomib, ixabepilone, lapatinib, larotrectinib, lenalidomide, lenvatinib, lorlatinib, marizomib, midostaurin, mitoxantrone, neratinib, netarsudil, nilotinib, nintedanib, niraparib, olaparib, oprozomib, osimertinib, paclitaxel, palbociclib, panobinostat, pazopanib, pemetrexed, pemigatinib, pexidartinib, ponatinib, pralsetinib, quizartinib, radotinib, regorafenib, ribociclib, ripretinib, rivastigmine, romidepsin, rucaparib, ruxolitinib, selpercatinib, selumetinib, sirolimus, sonidegib, sorafenib, sunitinib, talazoparib, tazemetostat, temsirolimus, tepotinib, tivozanib, tofacitinib, topotecan, trametinib, tucatinib, tucidinostat, upadacitinib, vandetanib, vemurafenib, venetoclax, vinorelbine, vismodegib, vorinostat, zanubrutinib and freebases, pharmaceutical salts and derivatives of these drug compounds.

This list of compounds, however, is not intended to limit the scope of the disclosure. In fact, the compound encapsulated within the liposome can be any poorly water-soluble amphipathic weak base or amphipathic weak acid. Also, poorly water-soluble compounds other than a pharmaceutical or medicinal agent are also encompassed by the present disclosure.

Typically, the terms weak base and weak acid, as used in the foregoing, respectively refer to compounds that are only partially protonated or deprotonated in water. Examples of protonable agents include compounds having an amino group, which can be protonated in acidic media, and compounds which are zwitterionic in neutral media and which can also be protonated in acidic environments. Examples of deprotonable agents include compounds having a carboxy group, which can be deprotonated in alkaline media, and compounds which are zwitterionic in neutral media and which can also be deprotonated in alkaline environments.

The foregoing implies that aqueous solutions of compounds being weak amphipathic acids or bases simultaneously comprise charged and uncharged forms of the compounds. Only the uncharged forms may be able to cross the liposomal membrane.

When a compound (i.e., active pharmaceutical ingredient) used in the present invention contains relatively basic or acidic functionalities, salts of such compounds are included in the scope of the disclosure. Salts can be obtained by contacting the neutral form of such compounds with a sufficient amount of the desired acid or base, either neat or in a suitable inert solvent. Examples of salts for relative acidic compounds of the disclosure include sodium, potassium, calcium, ammonium, organic amino, or magnesium salts, or similar salts. When compounds of the present disclosure contain relatively basic functionalities, acid addition salts can be obtained by contacting the neutral form of such compounds with a sufficient amount of the desired acid, either neat or in a suitable inert solvent. Examples of acid addition salts include those derived from inorganic acids, such as hydrochloric, hydrobromic, nitric, carbonic, monohydrogen carbonic, phosphoric, monohydrogenphosphoric, dihydrogenphosphoric, sulfuric, monohydrogensulfuric, hydriodic, or phosphorous acids, and the like, as well as the salts derived from organic acids, such as acetic, propionic, isobutyric, maleic, malonic, benzoic, succinic, suberic, fumaric, lactic, mandelic, phthalic, benzenesulfonic, p-toluenesulfonic, citric, tartaric, or methanesulfonic acid, and the like. Also included are salts of amino acids such as arginate and the like, and salts of organic acids like glucuronic or galacturonic acids and the like. Certain specific compounds of the present disclosure contain both basic and acidic functionalities that allow the compounds to be converted into either base or acid addition salts.

An exemplary compound is an organic molecule with a molecular weight between about 50 Da and 5000 Da and specifically in the range between 100 Da to 1000 Da.

### II. Determining In-Vitro Non-Antagonistic Drug Ratios

In a further embodiment of the disclosure, two or more drug compound can be encapsulated into liposomes at synergistic or additive (i.e., non-antagonistic) drug-to-drug ratios. At least one of such compounds is poorly water soluble. The therapeutically effective and non-antagonistic ratio of the compound is determined by assessing the biological activity or effects of the agents on relevant cell culture and/or tumor homogenates from individual patient biopsies, over a range of concentrations. Any method which results in determination of a ratio of the therapeutic agents which maintains a desired therapeutic effect may be used. For example, unless otherwise noted, the Chou-Talalay median-effect method was used in the examples disclosed in this disclosure (Chou, T. C., J. Theor. Biol., 1976, 39:253-276).

The underlying experimental data are generally determined in-vitro using cells in culture. Sometimes preferably, the combination index (CI), which is plotted as a function of the fraction of cells affected (Fa) is a surrogate parameter for concentration range. The term “fraction affected” refers to the faction of cells that is affected by a particular drug dose on their growth in an in vitro assay. Fraction affected is used to calculated combination index as described by Chou and Talalay procedure. Preferred combinations of agents are those that display synergy or additivity over a substantial range of Fa values. Combinations of agents are selected if non-antagonistic over at least about 5% of the concentration range wherein greater than 1% of the cells are affected, i.e., a Fa range greater than 0.01. Sometime preferably, a larger portion of overall concentration exhibits a favorable CI; for example, 5% of a Fa range of 0.2-1.0. Sometimes more preferably about 10% of this range exhibits a favorable CI. Sometimes even more preferably, about 20% of the Fa range, over about 50%, or over at least about 70% of the Fa range of 0.2 to 1.0 are utilized in the compositions. Combinations that display synergy over a substantial range of Fa values may be re-evaluated at a variety of agent ratios to define the optimal ratio to enhance the strength of the non-antagonistic interaction and increase the Fa range over which synergy is observed.

While it would be desirable to have synergy over the entire range of concentrations over which cells are affected, it has been observed that in many instances, the results are considerably more reliable in a Fa range of 0.2-0.8 when using a spectrophotometric method such as the MTT assay. Thus, although the synergy exhibited by combinations of the invention is set forth to exist within the broad range of 0.01 or greater, sometimes preferably the synergy is established in the Fa range of 0.2-0.8. Other more sensitive assays, however, can be used to evaluate synergy at Fa values greater than 0.8, for example, bioluminescence or clonogenicity assays.

The optimal combination ratio may be further used as a single pharmaceutical unit to determine synergistic or additive interactions with a third agent. In addition, a three-agent s combination may be used as a unit to determine non-antagonistic interactions with a fourth agent, and so on.

As set forth above, the in vitro studies on cell cultures will be conducted with “relevant” cells. The choice of cells will depend on the intended therapeutic use of the agent. Only one relevant cell line or cell culture type needs exhibit the required non-antagonistic effect in order to provide a basis for the compositions to come within the scope of the disclosure.

For example, in one preferred embodiment of the disclosure, the combination of agents is intended for anticancer therapy. In a frequent embodiment, the combination of agents is intended for multiple cancers, such as multiple myeloma, lung cancer, non-small cell lung cancer, leukemia or lymphoma therapy, breast cancer, triple negative breast cancer, gastrointestinal cancer, colorectal cancer, and renal cell carcinoma. Appropriate choices will then be made of the cells to be tested and the nature of the test. In particular, tumor cell lines are suitable subjects and measurement of cell death or cell stasis is an appropriate end point. As will further be discussed below, in the context of attempting to find suitable non-antagonistic combinations for other indications, other target cells and criteria other than cytotoxicity or cell stasis could be employed.

For determinations involving antitumor agents, cell lines may be obtained from standard cell line repositories (NCI or ATCC for example), from academic institutions or other organizations including commercial sources. Some preferred cell lines would include one or more selected from cell lines identified by the Developmental Therapeutics Program of the NCI/NIH. The tumor cell line screen used by this program currently identifies about 60 different tumor cell lines representing leukemia, melanoma, and cancers of the lung, colon, brain, ovary, breast, prostate, stomach, and kidney, etc. The required non-antagonistic effect over a desired concentration range need be shown only on a single cell type; however, sometimes preferably at least two cell lines, sometimes more preferably three cell lines, five cell lines, or even 10 cell lines, exhibit this effect. The cell lines may be established tumor cell lines or primary cultures obtained from patient samples. The cell lines may be from any species, but the preferred source will be mammalian and in particular human. The cell lines may be genetically altered by selection under various laboratory conditions.

In one preferred embodiment, the given effect (Fa) refers to cell death or cell stasis after application of a cytotoxic agent to a cell culture. Cell death or viability may be measured by MTT assay in this disclosure. Non-antagonistic ratios of two or more agents can be determined for disease indications other than cancer and this information can be used to prepare therapeutic formulations of two or more drugs for the treatment of these diseases. With respect to in-vitro assays, many measurable endpoints can be selected from which to define drug synergy, provided those endpoints are therapeutically relevant for the specific disease. As set forth above, the in-vitro studies on cell cultures will be conducted with “relevant” cells. The choice of cells will depend on the intended therapeutic use of the agent. In-vitro studies on individual patient biopsies or whole tumors can be conducted with “tumor homogenate.” generated from homogenization of the tumor sample(s) into single cells. In one preferred embodiment, the given effect (Fa) refers to cell death or cell stasis after application of a cytotoxic agent to a “relevant” cell culture. Cell death or viability may be measured using a number of the methods known in the art.

In one embodiment, one anthracycline (i.e., doxorubicin) and one proteasome inhibitor (i.e., carfilzomib) are combined for synergy and the synergistic drug-to-drug molar ratio was determined by the above-mentioned combination index (CI)-based method. The combination of anthracyclines and proteasome inhibitors is known to be synergistic. Specifically, doxorubicin and bortezomib have been shown to be synergistic and are FDA approved in combination for the treatment of multiple myeloma (Mitsiades, N., Blood, 2003, 101:2377-80). Carfilzomib, a second-generation proteasome inhibitor, has shown to have reduced off-target activity compared with bortezomib, and the former can eliminate the dose-limiting side-effects seen with bortezomib, such as peripheral neuropathy (Demo, S. D., Cancer Res., 2007, 67:6383-91). In a preferred embodiment, the molar ratio of carfilzomib and doxorubicin that shows a synergistic therapeutic effect is in the range between 1:50 to 1:1000.

In one embodiment, two protein kinase inhibitors, i.e., afatinib and dasatinib, are combined for synergy and the synergistic drug-to-drug molar ratio was determined by the CI-based method. The combination of afatinib and dasatinib may affect the SFK/FAK, PI3K/PTEN/Akt, Ras/Raf/MEK/ERK, and JAK/Stat signaling pathways, which can eventually reverse the drug resistance from the cancer cell when treated by either of the compound alone (Wang. M., Oncotarget, 2018, 9:16533-46). In a preferred embodiment, the molar ratio of afatinib and dasatinib that shows a synergistic therapeutic effect is in the range between 1:30 to 30:1.

In another embodiment, afatinib is combined with another protein kinase inhibitor, i.e., ceritinib, for synergy and the synergistic drug-to-drug molar ratio was determined by the CI-s based method. The combined inhibition on both Abl/Src family kinases (by dasatinib) and anaplastic lymphoma kinase (by ceritinib) can improve the treatment outcome (van Erp, A., Target Oncol., 2017, 12:815-826). In a preferred embodiment, the molar ratio of afatinib and ceritinib that shows a synergistic therapeutic effect is in the range between 1:30 to 30:1.

### III. Solubility Improving Agents

As noted hereinbefore, in exemplary embodiments of the disclosure a complex between a drug compound and a solubility improving agent (or sometimes called solubility enhancing agent, solubility enhancer, solubility enhancement agent, or the like) is added to the external aqueous medium of a liposome preparation to increase the rate and efficiency of the uptake of the poorly water-soluble agent from the external medium into the aqueous core of the liposome.

According to an embodiment of the present disclosure, a method as defined in the foregoing is provided using a solubility improving agent selected from the following: complexation agents, co-solvents, surfactants, and emulsifiers. The solubility improving agent typically increases the solubility of the poorly water-soluble agent in the external aqueous medium at least two-fold, preferably five-fold, and more preferably 10-fold.

Exemplary solubility improving agent include the following: a-, p-, and y-cyclodextrin and the cyclodextrin can be modified with alkyl-, hydroxyalkyl-, dialkyl-, and preferably sulfoalkylether modified cyclodextrins; Polyvinylpyrrolidone (povidone) with different molecular weight; Polyethylene glycol (PEG) with different molecular weight; Hydroxyl propyl methylcellulose (HPMC); Polyvinyl alcohol-polyethylene glycol graft-copolymer (Kollicoat® IR); Chitosan; Hydroxy propyl cellulose; Polyvinyl alcohol (PVA); Poly (2-hydroxy ethyl methacrylate); Methacrylic copolymers (Eudragit® S100 sodium salts and Eudragit® L100 sodium salts); Poloxamers. i.e., nonionic triblock copolymers composed of a central hydrophobic chain of polyoxypropylene (poly(propylene oxide)) flanked by two hydrophilic chains of polyoxyethylene (poly(ethylene oxide)); Polyglycolized glyceride (Labrasol); Polyoxyethylene sorbitan monoesters (Tweens); Sorbitan esters (Spans); Polyoxyethylene stearates; Poly (β-benzyl-L-aspartate)-β-poly(ethylene oxide); Poly(caprolactone)-b-poly(ethylene oxide); Ethanol: Dimethyl sulfoxide; Oleic acid, and combinations thereof.

This list of solubility improving agent, however, is not intended to limit the scope of the disclosure. In fact, any molecule that can increase the aqueous solubility of a poorly water-soluble compound is encompassed by the present disclosure.

In an exemplary embodiment, the solubility improving agent is selected from sodium salt of sulfobutylether-p-cyclodextrin, hydroxypropyl-p-cyclodextrin and polyvinylpyrrolidone.

### IV. Liposomes

The term “liposome” is used herein in accordance with its usual meaning, referring to nanometer sized lipid vesicles composed of a bilayer mainly composed of phospholipids or any similar amphipathic lipids encapsulating an internal aqueous medium. The liposomes of the present disclosure can be unilamellar vesicles such as small unilamellar vesicles (SUVs) and large unilamellar vesicles (LUVs), as well as multilamellar vesicles (MLVs). Typically, those liposomes exhibit a particle size in the range from 20 nm to 300 nm, and specifically in the range from 50 nm to 200 nm. No particular limitation is imposed on the liposomal membrane structure in the present disclosure. The term liposomal membrane refers to the bilayer of phospholipids separating the internal aqueous medium from the external aqueous medium.

Exemplary liposomal membranes useful in the current disclosure may be formed from a variety of vesicle-forming lipids, typically including dialiphatic chain lipids, such as phospholipids, diglycerides, dialiphatic glycolipids, single lipids such as sphingomyelin and glycosphingolipid, cholesterol and derivates thereof, and combinations thereof. As defined herein, phospholipids are amphiphilic agents having hydrophobic groups formed of long-chain alkyl chains, and a hydrophilic group containing a phosphate moiety. The group of phospholipids includes phosphatidic acid, phosphatidyl glycerols, phosphatidylcholines, phosphatidylethanola mines, phosphatidylinositols, phosphatidylserines, and mixtures thereof. Preferably, the phospholipids are chosen from hydrogenated egg yolk phosphatidylcholine (HEPC), soy phosphatidylcholine (SPC), egg yolk phosphatidylcholine (EYPC), 1,2-distearoyl-sn-glycero-3-phosphocholine (DSPC), hydrogenated soy phosphatidylcholine (HSPC), 1,2-dimyristoyl-sn-glycero-phophocholine (DMPC), 1,2-dioleoyl-sn-glycero-3-phosphocholine (DOPC), 1,2-diundecanoyl-sn-glycero-phosphocholine (DUPC), 1,2-dipalmitoyl-sn-glycero-3-phosphocholine (DPPC), 1,2-dioleoyl-snglycero-3-phospho-rac-(1-glycerol) sodium salt (DOPG), 1,2-dipalmitoyl-sn-glycero-3-phospho-rac-(1-glycerol) sodium salt (DPPG), 1,2-dioleoyl-sn-glycero-3-phosphoethanolamine (DOPE), 1-palmitoyl-2-oleoyl-sn-glycero-3-phosphocholine (POPC), 1-palmitoyl-2-oleoyl-sn-glycero-3-phosphoethanolamine (POPE), 1-palmitoyl-2-oleyol-sn-glycero-3-phospho-rac-(1-glycerol) sodium salt (POPG), N-(3-malimide-1-oxopropyl)-1,2-dioleoyl-sn-glycero-3-phosphoethanolamine (DOPE-mal), 1,2-dipalmitoyl-sn-glycero-3-phosphoethanolamine (DPPE), 1,2,-dimyristoyl-sn-glycero-3-phosphoethanolamine (DMPE), 1,2-distearoyl-sn-glycero-3-phosphoethanolamine (DSPE), 1,2-dilinoleoyl-sn-glycero-3-phosphocholine (DLPC), 1,2-diphytanoyl-sn-glycero-3-phosphoethanolamine, monomethyl-phosphatidylethanolamine, dimethyl-phosphatidylethanolamine, 1,2-dierucoyl-sn-glycero-3-phosphoethanolamine (DEPE). 1-stearoyl-2-oleoyl-sn-glycero-3-phosphoethanolamine (SOPE), 12-dilinoleoyl-sn-glycero-3-phosphocholine (DLOPC), 1,2-dilauroyl-sn-glycero-3-phosphocholine (DLPC), and others and mixtures thereof. Other diacyl-phosphatidylcholine, diacyl-phosphatidylethanolamine, and diacyl-phosphatidylserine phospholipids, sterol modified lipids, cationic lipids and zwitter lipids may also be used. In some embodiments, the acyl groups in these lipids are acyl groups derived from fatty acids having C10-C24 carbon chains (e.g., lauroyl, myristoyl, palmitoyl, stearoyl, or oleoyl).

Liposomal membranes according to the present disclosure may further comprise ionophores such as nigericin and A23187.

In the composition according to the present disclosure, an exemplary liposome phase transition temperature is between −20° C. and 100° C., and specifically in the range between 20° C. and 80° C. The phase transition temperature is the temperature required to induce a change in the physical state of the lipids constituting the liposome, from the ordered gel phase, where the hydrocarbon chains are fully extended and closely packed, to the disordered liquid crystalline phase, where the hydrocarbon chains are randomly oriented and fluid. Above the phase transition temperature of the liposome, the permeability of the liposomal membrane increases. Choosing phospholipids with a transition temperature that is higher than the exposed environment or the in-use conditions, could provide a non-leaking liposomal composition, i.e., the concentration of the poorly water-soluble agent in the internal aqueous medium is maintained during exposure to the environment. Alternatively, when high membrane permeability is needed, such as during remote drug loading process, the environmental temperature would be set to be higher than the lipid phase transition temperature, therefore the drug can diffuse through the lipid membrane and being encapsulated within the core of the liposome.

As is generally known in the art, phase transition temperatures of liposomes can, among other parameters, be influenced by the choice of phospholipids and by the addition of steroids. Sterols may be selected from the non-limiting list of lanosterol, stigmasterol, cholesterol, cholesterol derivatives, ergosterol, and ergosterol derivatives. Non-limiting examples of cholesterol derivatives include 5α-cholestanol, 5α-coprostanol, cholesteryl-(2′-hydroxy)ethyl ether, cholesteryl-(4′-hydroxy)butyl ether, 6-ketocholestanol, thiocholesterol, cholesteryl acetate, cholesteryl sulfate, cholestane-3,5-diene, 5α-coprostane, cholestenone, 5α-cholestanone, cholesteryl dodecanoate, and others and mixtures thereof. Therefore, in an embodiment of the disclosure, a method according to any of the foregoing is provided in which the liposomes comprise one or more components selected from different phospholipids and cholesterol in different molar ratios in order to modify the overall transition temperature of the liposome and the stability of the liposome during storage and in plasma. It is generally known in the art that less cholesterol in the lipid composition will result in less stable liposomes in plasma.

In an exemplary lipid composition of use in the disclosure, a phospholipid or combinations of phospholipids comprise at least 10 mol %, and specifically at least 30 mol % of the total lipid present in the liposome; and cholesterol or its derivatives comprises from 5 mol % to 50 mol % of the total lipid present in the liposome.

Polyethylene glycol (PEG)-lipid conjugates have been used extensively to improve circulation times for liposome-encapsulated therapeutic compounds, thereby to enhance the accumulation of the liposome in the desired disease site and to avoid the detection of liposomes by the body's immune system. One or more PEG-lipid conjugates can be incorporated into the liposome to exert the above-mentioned function. PEG conjugated lipids may be selected from the following non-limiting list of 1,2-distearoyl-sn-glycero-3-phosphoethanolamine-N-[methoxy(polyethylene glycol)-2000] sodium salt (mPEG2000-DSPE), 1,2-dipalmitoyl-sn-glycero-3-phosphoethanolamine-N-[methoxy(polyethylene glycol)-2000] sodium salt (PEG2000-DPPE), 1,2-dimyristoyl-rac-glycero-3-methoxypolyethylene glycol-2000 (PEG2000-DMG), distearoyl-rac-glycerol-PEG2000 (PEG2000-DSG), methoxypolyethyleneglycoloxy(2000)-N,N-ditetradecylacetamide (ALC-0159), 1,2-dioleoyl-sn-glycero-3-phosphoethanolamine-N-[amino(polyethylene glycol)-2000] sodium salt (DOPE-PEG1000-amine), 1,2-dioleoyl-sn-glycero-3-phosphoethanolamine-N-[amino(polyethylene glycol)-2000] sodium salt (DOPE-PEG2000-amine), 1,2-dioleoyl-sn-gly cero-3-phosphoethanolamine-N-[carboxy(polyethylene glycol)-1000] sodium salt (DOPE-PEG1000-COOH), 1,2-dioleoyl-sn-glycero-3-phosphoethanolamine-N-[carboxy(polyethylene glycol)-2000] sodium salt (DOPE-PEG2000-COOH), and cholesterol-(polyethylene glycol-600) (PEG600-Chol). PEGylated ceramides, PEGylated phosphatidic acids. PEGylated phosphatidylethanolamines, PEGylated dialkylamines, PEGylated diacylglycerols, PEGylated dialkylglycerols, PEGylated glycerides, PEGylated sterols, and others and mixtures thereof. In some embodiments, the polyethylene glycol chain will have an average molecular mass of 2000 atomic mass units. Other polymer conjugated lipid can also be used to extend the blood circulation time of the liposome. For example, polyglycerol-modified, polyacrylamide-modified, polydimethylacrylamide-modified, polyvinylpyrrolidone-modified, hyaluronic acid-modified, heparin-modified, polysialic acid-modified, etc.

In one embodiment, the polymer-conjugated lipid (e.g., PEG-lipid) that inhibits aggregation of liposomes comprising from 0 mol % to 10 mol % of the total lipid present in the liposome.

In one embodiment, the preferred PEG-lipid are mPEG2000-DSPE and PEG2000-DMG.

For liposome preparation, multilamellar vesicles with relatively large particle size and wide size distributions are usually prepared first through a lipid hydration step, wherein the selected lipids are hydrated and dispersed in aqueous solution. Following the hydration step, a size reduction process is generally performed to decrease the particle size and narrow the size distribution to the desired range. Also, the liposome lamellarity can be decreased after the size reduction step, in other words, unilamellar vesicles can be generated through this process. A size range of about 20-200 nm allows the liposome suspension to be sterilized by filtration through a conventional filter, typically a 0.22- or 0.45-micron filter. Several techniques known to the art are available for size reduction on liposomes. For example, sonicating a liposome suspension either by bath or probe sonication produces a progressive size reduction down to small unilamellar vesicles (SUVs) less than about 50 nanometers in size. Homogenization is another method which relies on shearing energy to fragment large liposomes into smaller ones. In a typical homogenization procedure, multilamellar vesicles are processed through a homogenizer at defined pressure through multiple cycles until desired liposome size is reached, typically between about 50 nm and 500 nm. Extrusion of liposome through a small-pore polycarbonate membrane or an asymmetric ceramic membrane is also an effective method for reducing liposome sizes to a relatively well-defined size distribution. Typically, the liposome suspension is extruded through the membrane with defined pore size one or more times until the desired liposome size and size distribution is achieved. The liposomes may be extruded through successively smaller-pore membranes, to achieve a gradual reduction in liposome size. Alternatively limit size liposomes can be prepared using microfluidic techniques wherein the lipid in an organic solvent, such as ethanol or ethanol-aprotic solvent mixtures is rapidly mixed with the aqueous medium, so that the organic solvent/water ratio is less than 30%, in a microchannel with dimensions less than 300 microns and preferable less than 150 microns in wide and 50 microns in height. The organic solvent is then removed from the liposomes by dialysis. Other useful sizing methods such as reverse phase evaporation and freeze/thaw method are known to those of skill in the art.

Exemplary liposomes for use in various embodiments of the disclosure have a size from about 20 nm to about 50 microns. In an exemplary embodiment, the liposomes are from about 30 nm to about 150 nm in diameter.

In certain processes, control of temperature may be important or critical to achieving the desired results. For example, during liposome hydration and particle size reduction step, temperature should be higher than the phase transition temperature of all the lipids used in the liposome formulation. In some embodiments, the temperature during liposome hydration and particle size reduction is at least or higher than 50° C.

### V. Remote Loading for Poorly Water-Soluble Compounds

The internal aqueous medium typically is the original medium in which the liposomes were prepared during the hydration step, and which initially becomes encapsulated upon formation of the liposome. This original medium contains one or a mixture of trapping agent(s) that are used for the remote loading of drug compounds. The original medium may also contain one or a mixture of buffering agent(s) that are used to maintain the liposome internal pH. Following the liposome size reduction step, a medium exchange process is used to replace the external liquid medium with medium of a different composition. Several techniques are known in the art for this purpose, for example, dialysis, ultracentrifugation, size exclusion chromatography and others. After the medium exchange step, the original medium is still encapsulated within the internal aqueous core, but the external liquid medium is changed to other compositions, e.g., other charged species. Thereby, an ion gradient can be generated across the liposome bilayer membrane. Such processed liposomes with transmembrane ion gradient, i.e., a gradient of trapping agent, can then be used for the remote loading of therapeutic compounds. The terms active-loading and remote-loading are synonymous and can be used interchangeably.

During active loading, the complex formed between the solubility improving agent and the poorly water-soluble agent can facilitate the transfer of the compound from external aqueous medium across the liposome membrane to the internal aqueous medium. Once located within the internal aqueous core, the compound can interact with the trapping agent to form a complex through various interactions, e.g., ionic interaction, hydrophobic interaction, hydrogen bonding, and such newly formed complex can serve as the driving force to further recruit more drugs to be loaded within the liposome through active loading process.

The pH value in the liposome external liquid medium was surprisingly found to affect drug encapsulation efficiency during active drug loading. An optimal external pH range is needed to achieve high drug encapsulation efficiency. Without wishing to be bound by any particular theory, it is believed that loading performed at an external pH lower than the optimal range leads to the formation of high content of ionized compound in the external medium which may impede the transmembrane drug diffusion, since only the neutral form of the compound can freely pass through the lipid membrane. On the other hand, loading performed at an external pH higher than the optimal range suffers from drug solubility issue even in the presence of a solubility improving agent, and in this case the poorly water-soluble drug cannot be sufficiently dissolved within the external medium; thereby a low drug encapsulation efficiency is obtained.

The optimal drug loading pH in the external medium for each compound will depend on both the physicochemical properties for the specific drug compound of interest (e.g., pKa), and also the selected solubility improving agent of use. In an exemplary embodiment, after active loading step, about 90% or greater of the agent is encapsulated in the aqueous compartment of the liposome and about 10% or less of the agent is in a complex with the solubility improving agent located external to the liposome.

For the remote loading of poorly water-soluble weak amphipathic base, a pH gradient is established across the lipid membrane. It was surprisingly observed that the pH value in the liposome external medium is required to be lower than that of the internal medium. This is completely opposite to the conventional pH gradient approach used for the remote loading of weak bases reported previously (Madden. D., Chemistry and Physics of Lipids, 1990, 53:37-46), wherein the pH value in the external medium is higher than that of the internal medium. Without wishing to be bound by any particular theory, the pH gradient requirement presented in this disclosure is determined by considering the degree of compound ionization in the external medium, compound solubility as well as the drug and lipid degradation induced by acidic or basic conditions.

In an exemplary embodiment for the active loading of poorly water-soluble weak amphipathic base, the pH value of the external medium is 8.0 or 7.0 or 6.0 or 5.0 or 4.0 or 3.0 s or 2.0 or 1.0 or 0.5 or 0.25 or 0.1 unit lower than that of the internal medium. In one embodiment, the internal medium pH is between 5.0 to 10.0 and the external medium pH is between 2.0 to 5.0.

In one embodiment, one or a mixture of buffering agent(s) is added into the aqueous solution during the lipid hydration step when preparing the liposome, therefore such buffering agent(s) stays within the internal aqueous core of the liposome during drug loading step to maintain the intraliposomal pH and to avoid significant internal pH fluctuation upon drug encapsulation. Exemplary buffering agents are the following: acetic acid, citric acid, histidine, HEPES, lactic acid, succinic acid, phosphate salt, tromethamine (Tris) and others.

In one embodiment, no buffering agent is included in the aqueous solution during the lipid hydration step when preparing the liposome. In such a case, there is no buffering agent within the interior aqueous core of the liposome.

In one embodiment, the preferred pH of external medium for the active loading of carfilzomib is in the range from 3.0 to 5.0, and the preferred pH of internal medium is in the range from 5.0 to 10.0.

Regarding the distribution (external, internal or both) of the solubility improving agent within the liposome after drug loading, it highly depends on the physicochemical properties of the molecule of use, e.g., molecular weight, lipid membrane permeability, pKa and charge. The external medium pH during drug loading can affect the above properties of the solubility improving agent. In one embodiment wherein sodium salt of sulfobutylether-β-cyclodextrin (SBE-β-CD) is used as the solubility improving agent, without wishing to be bound to any particular theory, it is believed that when the external pH is less than its pKa, then majority of the sulfo group on SBE-β-CD is in neutral form, thereby the molecule can carry the poorly water-soluble compound and diffuse together into the liposome interior core as the drug/cyclodextrin complex. In this case, both inside and outside the liposome should contain SBE-β-CD.

In another embodiment wherein sodium salt of SBE-β-CD is used as the solubility improving agent, but the external medium pH during drug loading is higher than its pKa, then majority of the sulfo group on SBE-β-CD is negatively charged which can impede its permeation through the lipid membrane. In this case, SBE-β-CD should only stay outside the liposome.

Trapping agents used in active drug loading establish the transmembrane pH and ion s gradient and also may form precipitation, aggregation, or gelation with the therapeutic agent thereby retain the compound within the liposome. Suitable trapping agents may be anionic, cationic, amphoteric, or nonionic active agents including, but are not limited to those containing carboxylate, polyphosphate, sulfonate including long chain alkyl sulfonates and alkyl aryl sulfonates and sulfate. Cationic trapping agents include quaternary ammonium compounds such as benzalkonium chloride, benzethonium chloride, cetrimonium bromide, stearyl dimethylbenzyl ammonium chloride, polyoxyethylene and coconut amine, and the like.

More specific examples of trapping agents include ammonium sulfate, salt metals and ammonium or substituted ammonium salts of the following: polyanionized sulfated cyclodextrin, sulfobutyl ether cyclodextrin, polyanionized sulfated sugar, polyphosphates, and the like.

Specifically, trapping agents include ammonium or substituted ammonium salts of the following polyanionized sulfated sugars: sucrose octasulfate, chondroitin sulfate, dermatan sulfate, keratan sulfate, heparan sulfate and sulfated hyaluronic acid, fucoidan, galactan, carrageenan, rhamnan sulfate, galactofucan, mannoglucuronofucan, arabinogalactans sulfate, mannan sulfate, sulfated heterorhamnan and xylomannan sulfate, and the like.

Specifically, trapping agents include ammonium or substituted ammonium salt of the following forms of sulfobutylether cyclodextrin: sulfobutylether-a-cyclodextrin, sulfobutylether-β-cyclodextrin, and sulfobutylether-γ-cyclodextrin, and the like.

Specifically, trapping agents include ammonium or substituted ammonium salts of the following polyphosphate: phytic acid, triphosphoric acid, polyphosphoric acid and cyclic trimetaphosphate.

Specifically, the counter ion to the above polyanions includes ammonium and substituted ammonium which further includes the protonated form of the following: triethylamine, triethanolamine, tris(hydroxymethyl)aminomethane or tromethamine, diethanolamine, ethylenediamine, tributylamine, 1,4-diazabicyclo[2.2.2]octane, diethylethanolamine, diethanolethylamine, ethanolamine, morpholine, and the like.

Metal-based trapping agents include the salt form of the following: ions of calcium, copper, zinc, magnesium, manganese, nickel, and cobalt. The counter ion to the metal includes acetate, carbonate, citrate, sulfate, chloride, halide, gluconate, bromide, and hydroxide.

More specifically, trapping agents used for the liposome loading of weak amphipathic s bases include the following: ammonium sulfate, triethylammonium sucrose octasulfate (TEA-SOS), triethylammonium sulfobutylether-β-cyclodextrin (TEA-SBE-β-CD); tris(hydroxymethyl)aminomethane salt of sulfobutylether-β-cyclodextrin (Tris-SBE-β-CD), triethylammonium salt of phytic acid or inositol hexaphosphate (TEA-IP6), copper gluconate, copper sulfate, copper chloride and zinc sulfate.

Specifically, trapping agents used for the liposome loading of weak amphipathic acids include the following: calcium acetate, magnesium acetate and zinc acetate.

One or more therapeutic drug compound can be remotely loaded within the liposome. At least one of such agents is poorly water-soluble. Encapsulation of drug combinations can be performed in the same liposome vehicle or in separate liposomes. In the former case, multiple compounds for combination can be added at the same time into the liposome for drug loading. For example, both drug A and drug B can be dissolved together in the presence of solubility improving agent(s) as aqueous solution, and then the drug solution is mixed with liposome for loading. Alternatively, drug compounds can also be sequentially loaded into the same liposome to avoid possible interference on loading efficiency and/or concerns related to drug degradation. For example, drug A can be solubilized in the presence of solubility improving agent first as aqueous solution, and then is mixed with liposome for loading. After a defined amount of time, drug B is then added into the same liposome pre-loaded with drug A for combined drug loading.

When encapsulation of drugs into separate liposomes is desired, the lipid composition of each liposome may be quite different to allow for coordinated pharmacokinetics. By altering the liposome vehicle composition, release rates of encapsulated drugs can be matched to allow desired ratios of the drugs to be delivered to the tumor site. Means of altering release rates include increasing the acyl chain length of vesicle forming lipids to improve drug retention, controlling the exchange of surface grafted hydrophilic polymers such as polyethylene glycol group on mPEG-DSPE out of the liposome membrane, and incorporating membrane-rigidifying agents such as sterols or sphingomyelin into the membrane. It should be apparent to those skilled in the art that if a first and second drug are desired to be administered at a specific drug ratio and if the second drug is retained poorly within the liposome composition of the first drug (e.g., DMPC/Chol), that improved pharmacokinetics may be achieved by encapsulating the second drug in a liposome composition with lipids of increased acyl chain length (e.g., DSPC/Chol). When encapsulated in separate liposomes, it should be readily accepted that ratios of both drugs that have been determined on a patient-specific basis to provide optimal therapeutic activity can be generated for individual patients by combining the appropriate amounts of each liposome encapsulated drug prior to administration.

The temperature control during drug loading step is critical to ensure the compound can efficiently permeate through the lipid membrane of the liposome, and such temperature should be higher than the phase transition temperature of all the lipids used in the liposome formulation. In a preferred embodiment, the drug loading temperature is higher than 50° C.

After the drug loading step, a free drug removal process can be optionally employed to remove the unencapsulated drug compound from the external medium of the liposome suspension. Techniques for free drug removal is similar to those used in the previous medium exchange process, i.e., dialysis, ultracentrifugation, size exclusion chromatography and others. In the final external medium of the liposome suspension, a buffering agent and a tonicity modifier are commonly included. Exemplary buffering agents are acetic acid, citric acid, histidine, HEPES, lactic acid, succinic acid, phosphate salt, tromethamine (Tris) and others. The pH value of the final external medium of the liposome suspension is between 5.0 to 10.0, preferably between 6.0 to 8.0. When the pH of the final external medium of the liposome suspension is less than 5.0 or is higher than 10.0, severe lipid degradation in the product can happen in a short time when stored as liquid. Therefore, close to neutral pH of the final external medium is preferred. Exemplary tonicity modifier includes sucrose, dextrose, mannitol, trehalose, sodium chloride and others.

In an exemplary embodiment, after free drug removal step, about 98% or greater of the agent is encapsulated in the aqueous compartment of the liposome and less than 2% of the agent is located external to the liposome.

To ensure an acceptable product stability, in the final manufacturing step, liposomes are optionally dehydrated under reduced pressure using standard freeze-drying (lyophilization) equipment or equivalent apparatus. In various embodiments, the liposomes and their surrounding medium are frozen at low temperatures (e.g., −20 to −80° C.) before being dehydrated and placed under reduced pressure. To ensure that the liposomes will survive the dehydration process without losing a substantial portion of their internal contents, one or more protective sugars are typically employed to interact with the lipid vesicle membranes and keep them intact as the water in the system is removed. A variety of sugars can be used, including such sugars as trehalose, maltose, sucrose, glucose, lactose, and dextran. In general, disaccharide sugars have been found to work better than monosaccharide sugars, with the disaccharide sugars trehalose and sucrose being most effective. Other more complicated sugars can also be used. For example, aminoglycosides, including streptomycin and dihydrostreptomycin, have been found to protect liposomes during dehydration. Typically, one or more sugars are included as part of either the internal or external media of the lipid vesicles. Most preferably, the sugars are included in both the internal and external media so that they can interact with both the inside and outside surfaces of the liposomes' membranes. Inclusion in the internal medium is accomplished by adding the sugar or sugars to the buffer which becomes encapsulated in the lipid vesicles during the liposome formation process, i.e., lipid hydration step. In these embodiments the external medium used during the active loading process should also preferably include one or more of the protective sugars.

In one embodiment, the drug loaded liposomes can be prepared according to the following steps.
h) forming a lipid dispersion in a solution comprising said trapping agent(s), e.g., TEA-SOS and optionally a buffering agent(s), e.g., HEPES:
i) reducing liposome particle size at an elevated temperature (e.g., at or above 50° C.) by extruding the liposome through polycarbonate membranes with defined pore diameter, e.g., 100 nm;
j) substantially removing the trapping agent outside of the liposome thereby obtaining unloaded liposome, e.g., through a dialysis process;
k) separately, dissolving active pharmaceutical ingredient(s) in the presence of solubility improving agent(s) in aqueous solution. When two or more compounds are needed to be encapsulated, at least one of the drugs is poorly water soluble;
l) incubating said unloaded liposome with said drug or combined drug solution comprising solubility improving agent(s) at an elevated temperature (e.g., at or above 50° C.), thereby forming the drug loaded liposomes through active loading;
m) optionally, removing unloaded drug(s) and the solubility improving agent(s) outside the liposomes by dialysis or ultracentrifugation or size exclusion chromatography; and
n) optionally, forming dry form of the liposome product by lyophilization

### VI. Method of Treatment

In one aspect, the disclosure provides a method of treating a proliferative disorder, e.g., a cancer, in a subject, e.g., a human, the method comprising administering a composition that comprises a pharmaceutical formulation of the disclosure to a subject in an amount effective to treat the disorder, thereby treating the proliferative disorder.

In one embodiment, the pharmaceutical formulation is administered in combination with one or more additional anticancer agent, e.g., a chemotherapeutic agent or combination of chemotherapeutic agents described herein, and radiation.

In one embodiment, the cancer is a cancer described herein. For example, the cancer can be a cancer of the bladder (including accelerated and metastatic bladder cancer), breast (e.g., estrogen receptor positive breast cancer, estrogen, receptor negative breast cancer; HER-2 positive breast cancer; HER-2 negative breast cancer, progesterone receptor positive breast cancer, progesterone receptor negative breast cancer; estrogen receptor negative, HER-2 negative and progesterone receptor negative breast cancer (i.e., triple negative breast cancer); inflammatory breast cancer), colon (including colorectal cancer), kidney (e.g., transitional cell carcinoma), liver, lung (including Small and non-Small cell lung cancer, lung adenocarcinoma and Squamous cell cancer), genitourinary tract, e.g., ovary (including fallopian tube and peritoneal cancers), cervix, prostate, testes, kidney, and ureter, lymphatic system, rectum, larynx, pancreas (including exocrine pancreatic carcinoma), esophagus. Stomach, gall bladder, thyroid, skin (including squamous cell carcinoma), brain (including glioblastoma multiforme), head and neck (e.g., occult primary), and Soft tissue (e.g., Kaposi's sarcoma (e.g., AIDS related Kaposi's sarcoma), leiomyosarcoma, angiosarcoma, and histiocytoma).

In an exemplary embodiment, the cancer is multiple myeloma or a solid tumor. In one embodiment, the pharmaceutical formulation of the disclosure includes carfilzomib as the poorly water-soluble therapeutic agent.

In one aspect, the disclosure features a method of treating a disease or disorder associated with inflammation, e.g., an allergic reaction or an autoimmune disease, in a subject, e.g., a human, the method comprises: administering a composition that comprises a pharmaceutical formulation of the disclosure to a subject in an amount effective to treat the disorder, to thereby treat the disease or disorder associated with inflammation.

In one embodiment, the disease or disorder associated with inflammation is a disease or disorder described herein. For example, the disease or disorder associated with inflammation s can be for example, multiple sclerosis, rheumatoid arthritis, psoriatic arthritis, degenerative joint disease, spondyloarthropathies, gouty arthritis, systemic lupus erythematosus, juvenile arthritis, rheumatoid arthritis, osteoarthritis, osteoporosis, diabetes (e.g., insulin dependent diabetes mellitus or juvenile onset diabetes), menstrual cramps, cystic fibrosis, inflammatory bowel disease, irritable bowel syndrome, Crohn's disease, mucous colitis, ulcerative colitis, gastritis, esophagitis, pancreatitis, peritonitis, Alzheimer's disease, shock, ankylosing spondylitis, gastritis, conjunctivitis, pancreatitis (acute or chronic), multiple organ injury syndrome (e.g., secondary to septicemia or trauma), myocardial infarction, atherosclerosis, stroke, reperfusion injury (e.g., due to cardiopulmonary bypass or kidney dialysis), acute glomerulonephritis, vasculitis, thermal injury (i.e., sunburn), necrotizing enterocolitis, granulocyte transfusion associated syndrome, and/or Sjogren's syndrome. Exemplary inflammatory conditions of the skin include, for example, eczema, atopic dermatitis, contact dermatitis, urticaria, scleroderma, psoriasis, and dermatosis with acute inflammatory components. In some embodiments, the autoimmune disease is an organ-tissue autoimmune diseases (e.g., Raynaud's syndrome), scleroderma, myasthenia gravis, transplant rejection, endotoxin shock, sepsis, psoriasis, eczema, dermatitis, multiple sclerosis, autoimmune thyroiditis, uveitis, systemic lupus erythematosus, Addison's disease, autoimmune polyglandular disease (also known as autoimmune polyglandular syndrome), or Grave's disease.

In another embodiment, a pharmaceutical formulation of the disclosure or method described herein may be used to treat or prevent allergies and respiratory conditions, including asthma, bronchitis, pulmonary fibrosis, allergic rhinitis, oxygen toxicity, emphysema, chronic bronchitis, acute respiratory distress syndrome, and any chronic obstructive pulmonary disease (COPD). The pharmaceutical formulation of the disclosure, particle or composition may be used to treat chronic hepatitis infection, including hepatitis B and hepatitis C.

In one aspect, the disclosure features a method of treating cardiovascular disease, e.g., heart disease, in a subject, e.g., a human, the method comprising administering a pharmaceutical formulation of the disclosure to a subject in an amount effective to treat the disorder, thereby treating the cardiovascular disease.

In one embodiment, cardiovascular disease is a disease or disorder described herein. For example, the cardiovascular disease may be cardiomyopathy or myocarditis: Such as idiopathic cardiomyopathy, metabolic cardiomyopathy, alcoholic cardiomyopathy, drug-induced cardiomyopathy, ischemic cardiomyopathy, and hypertensive cardiomyopathy. Also, treatable or preventable using a pharmaceutical formulation of the disclosures, particles, compositions and methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. Other vascular diseases that can be treated or prevented include those related to platelet aggregation, the retinal arterioles, the glomerular arterioles, the Vasa nervorum, cardiac arterioles, and associated capillary beds of the eye, the kidney, the heart, and the central and peripheral nervous systems. Yet other disorders that may be treated with pharmaceutical formulation of the disclosure, include restenosis, e.g., following coronary intervention, and disorders relating to an abnormal level of high-density and low-density cholesterol.

In one embodiment, the pharmaceutical formulation of the disclosure can be administered to a subject undergoing or who has undergone angioplasty. In one embodiment, the pharmaceutical formulation of the disclosure, particle or composition is administered to a subject undergoing or who has undergone angioplasty with a stent placement. In some embodiments, the pharmaceutical formulation of the disclosure, particle or composition can be used as a strut of a stent or a coating for a stent.

In one aspect, the disclosure provides a method of treating a disease or disorder associated with the kidney, e.g., renal disorders, in a subject, e.g., a human, the method comprises: administering a pharmaceutical formulation of the disclosure to a subject in an amount effective to treat the disorder, thereby treating the disease or disorder associated with kidney disease.

In one embodiment, the disease or disorder associated with the kidney is a disease or disorder described herein. For example, the disease or disorder associated with the kidney can be for example, acute kidney failure, acute nephritic syndrome, analgesic nephropathy, atheroembolic renal disease, chronic kidney failure, chronic nephritis, congenital nephrotic syndrome, end-stage renal disease, good pasture syndrome, interstitial nephritis, kidney damage, kidney infection, kidney injury, kidney Stones, lupus nephritis, membranoproliferative GNI, membranoproliferative GN II, membranous nephropathy, minimal change disease, necrotizing glomerulonephritis, nephroblastoma, nephrocalcinosis, nephrogenic diabetes insipidus, nephrosis (nephrotic Syndrome), polycystic kidney disease, post-streptococcal GN, reflux nephropathy, renal artery embolism, renal artery stenosis, renal papillary necrosis, renal tubular acidosis type I, renal tubular acidosis type II, renal under perfusion, renal vein thrombosis.

In an exemplary embodiment, the disclosure provides a method of treating metal toxicity or metal overload. Examples of diseases or disorders associated with metal include iron overload disorders (e.g., thalassemia or sickle cell anemia), copper overload disorders (e.g., Wilson's disease), and radioisotope contamination (e.g., occurring subsequent to contamination with plutonium, uranium and other radio isotopes).

### Definitions

Unless defined otherwise, all terms of art, notations and other scientific terms or terminology used herein have the same meaning as is commonly understood by one of ordinary skill in the art to which this disclosure belongs. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a,” “an,” and “the” include plural reference, and vice versa, any plural forms include singular reference, unless the context clearly dictates otherwise.

The term “about” or “approximately”, unless otherwise defined, generally includes up to plus or minus 10% of the indicated number. For example, “about 10%” may indicate a range of 9% to 11%, and “about 20” may mean from 18 to 22. Sometimes preferably, “about” includes up to plus or minus 5% of the indicated value. Alternatively, “about” includes up to plus or minus 5% of the indicated value. When “about” is used before a range, it is applicable to both the lower end and the upper end of a range.

The term “substantially” as herein used means “for the most part” or “essentially,” as would be understood by a person of ordinary skill in the art, and if measurable quantitatively, refers to at least 90%, preferably at least 95%, more preferably at least 98%.

The terms “comprising”, “having”, “including”, and “containing”, or the like, are to be construed as open-ended terms (i.e., meaning “including, but not limited to”) unless otherwise noted.

As used herein, the term “synergistic effect” means an interaction between two or more drugs that causes the total effect of the drugs to be greater than the sum of the individual effects of each drug.

By “synergistic ratio” is meant the molar ratio of two or more drugs used in combination at which a synergistic effect can be obtained.

As used herein, the term “synergistic cytotoxic effect” refers an interaction between two or more drugs that causes the total effect of the drugs to be greater than the sum of the individual effects of each drug. This total effect results in cell kill and eventual tumor shrinkage.

As used herein, the term “synergistic cytostatic effect” refers an interaction between two or more drugs that causes the total effect of the drugs to be greater than the sum of the individual effects of each drug. This total effect results in tumor growth inhibition without direct cell killing.

The term “additive effect” means the combined effect produced by the action of two or more drugs, being equal to the sum of their separate effects.

By “additive ratio” is meant the molar ratio of the two or more drugs used in combination at which an additive effect can be obtained.

The term “non-antagonistic ratio” refers to both synergistic and additive ratio.

As used herein, the term “antagonistic effect” means a therapeutic response to exposure to two or more drugs that is less than would be expected if the known effects of the individual drugs were added together.

The term “antagonistic ratio” as used herein refers to molar ratio of two or more drugs used in combination at which an antagonistic effect can be obtained.

The term “combination index” refers to a parameter that is used to determine the degree of drug interaction. Combination Index (CI) can be calculated based on the median-effect analysis algorithm as described by Chou and Talalay (T. C. Chou and P. Talalay, Adv. Enzyme Reg., 1984, 22:27-55). A CI value <0.9 indicates synergistic drug interactions; 0.9≤CI≤1.1 reflects additive effect, and a CI>1.1 indicates antagonistic effect.

The term “fraction affected” refers to the faction of cells that is affected by a particular drug dose on their growth in an in vitro assay. Fraction affected is used to calculated combination index as described by Chou and Talalay procedure.

By “relevant” cells refer to at least one cell culture or cell line which is appropriate for testing the desired biological effect. As these agents are used as antineoplastic agents, “relevant” cells are those of cell lines identified by the Developmental Therapeutics Program (DTP) of the National Cancer Institute (NCI)/National Institutes of Health (NIH) as useful in their anticancer drug discovery program. Currently the DTP screen utilizes 60 different human tumor cell lines. The desired activity on at least one of such cell lines would need to be demonstrated.

By “tumor homogenate” refers to cells generated from the homogenization of patient biopsies or tumors. Extraction of whole tumors or tumor biopsies can be achieved through standard medical techniques by a qualified physician and homogenization of the tissue into single cells can be carried out in the laboratory using a number of methods well-known in the art.

The term “trapping agent” as used herein refers to a chemical compound that is presented within the aqueous compartment of the liposome and is used to entrap and retain one or more drugs within the same location inside of the liposome.

The term “poorly water-soluble” means being insoluble or having a very limited solubility in water, more in particular having an aqueous solubility of less than 1 mg/mL. As used herein, water solubilities refer to compound solubility measured at ambient temperature, which is typically about 20-25° C. In an exemplary embodiment, the water solubility of the compound is measured at about pH=7.0.

The terms “weak base” and “weak acid”, as used in the foregoing, respectively refer to compounds that are only partially protonated or deprotonated in water. Examples of protonable agents include compounds having an amino group, which can be protonated in acidic media, and compounds which are zwitterionic in neutral media and which can also be protonated in acidic environments. Examples of deprotonable agents include compounds having a carboxy group, which can be deprotonated in alkaline media, and compounds which are zwitterionic in neutral media and which can also be deprotonated in alkaline environments.

The term “zwitterionic” refers to compounds that can simultaneously carry a positive s and a negative electrical charge on different atoms.

The term “amphipathic”, as used in the disclosure typically employed to refer to compounds having both lipophilic and hydrophilic moieties.

The term “complexing agents,” or the like, as used herein, are solubility enhancing agents, which are water-soluble compounds that form water-soluble inclusion complexes with the poorly water-soluble agent, hence increasing the aqueous solubility of the poorly water-soluble compound.

The term “encapsulation efficiency” is defined by the equation below:

$$Encapsulation efficiency \% = \frac{Entrapped drug amount}{Entrapped drug amount + Unencapsulated drug amount} \times 100 \%$$

An “effective amount” or “an amount effective” refers to an amount of the pharmaceutical formulation of the disclosure which is effective, upon single or multiple dose administrations to a subject, in treating a cell, or curing, alleviating, relieving or improving a symptom of a disorder. An effective amount of the composition may vary according to factors such as the disease state, age, sex, and weight of the individual, and the ability of the compound to elicit a desired response in the individual. An effective amount is also one in which any toxic or detrimental effects of the composition are outweighed by the therapeutically beneficial effects.

As used herein, the term “prevent” or “preventing” as used in the context of the administration of an agent to a subject, refers to subjecting the subject to a regimen, e.g., the administration of a pharmaceutical formulation of the disclosure such that the onset of at least one symptom of the disorder is delayed as compared to what would be seen in the absence of the regimen.

As used herein, the term “subject,” “patient,” or the like, is intended to include human and non-human animals. Exemplary human subjects include a human patient having a disorder, e.g., a disorder described herein, or a normal subject. The term “non-human animals' includes all vertebrates, e.g., non-mammals (such as chickens, amphibians, reptiles) and mammals. Such as non-human primates, domesticated and/or agriculturally useful animals, e.g., sheep, dog, cat, cow, pig, etc.

As used herein, the term “treatment” of, or “treat” or “treating,” a subject having a disorder refers to subjecting the subject to a regimen, e.g., the administration of a s pharmaceutical formulation of the disclosure such that at least one symptom of the disorder is cured, healed, alleviated, relieved, altered, remedied, ameliorated, or improved. Treating includes administering an amount effective to alleviate, relieve, alter, remedy, ameliorate, improve, or affect the disorder or the symptoms of the disorder. The treatment may inhibit deterioration or worsening of a symptom of a disorder.

The term “pharmaceutically acceptable” describes a material that is not biologically or otherwise undesirable, i.e., without causing an unacceptable level of undesirable biological effects or interacting in a deleterious manner.

The term “liposome lamellarity” refers to the numbers of lipid bilayers in liposomes which influences the encapsulation efficiency and the drugs release kinetics.

The term “liposome” is used herein in accordance with its usual meaning, referring to nanometer sized lipid vesicles composed of a bilayer membrane mainly composed of phospholipids or any similar amphipathic lipids encapsulating an internal aqueous medium.

The liquid medium outside of the bilayer membrane where the liposome is suspended within is referred to as the external liquid medium.

The phrase “unilamellar vesicles” as used herein refers to spherical vesicles comprised of one lipid bilayer membrane which defines a single closed aqueous compartment. The bilayer membrane is composed of two layers of lipids: an inner layer and an outer layer. Lipid molecules in the outer layer are oriented with their hydrophilic head portions towards the external aqueous environment and their hydrophobic tails pointed downward toward the interior of the liposome. The inner layer of the lipid lays directly beneath the outer layer, the lipids are oriented with their heads facing the aqueous interior of the liposome and their tails towards the tails of the outer layer of lipid.

The phrase “multilamellar vesicles” as used herein refers to liposomes that are composed of more than one lipid bilayer membrane, which membranes define more than one closed aqueous compartment. The membranes are concentrically arranged so that the different membranes are separated by aqueous compartments, much like an onion.

The term “total lipid” refers to all the lipids and lipid derivatives used in the formulation, which include phospholipids (e.g., HSPC, DSPC, DPPC. DMPC and DSPG), sterol (e.g., cholesterol), and phospholipid conjugated with polyethylene glycol (e.g., mPEG-DSPE).

By “release” is meant that the drug encapsulated in a liposome passes through the lipid s membrane constituting the liposome and then exits to the outside of the liposome.

The term “encapsulation” as used herein, refers to encircling an internal phase typically resulting in an interior cavity separated from an external media. The components of the internal phase/interior cavity are thus “encapsulated” as described herein. As described herein, the encircled, or encapsulated, internal phase is the lipid bilayers and the aqueous phases. The amount of the therapeutic drug that is loaded into the interior cavity of the liposome and therefore unavailable to the external media until the liposome is triggered from release would be considered as “encapsulated” within the liposome.

The phrase “co-encapsulation” and “co-encapsulated” as used herein, refers to the situation where two or more therapeutic agents are encapsulated within the liposome.

The phrase “active loading” or “remote loading” as used herein refers to a drug loading technique used in liposome drug product preparation. The commonly used active loading methods in the art include the transmembrane pH gradient loading technique and transition metal loading technique. The former one utilizes an ammonium or a substituted ammonium salt of monoanion or polyanions as the trapping agent which is pre-loaded into the liposome prior to the encapsulation of therapeutic agent. Based on the equilibrium as determined by the pH gradient, the therapeutic agent can “actively” diffuse into the aqueous compartment of the liposome, interact with the pre-loaded trapping agent through the formation of precipitation, aggregation, or gelation, which serves as another driving force to encapsulate the therapeutic agent inside the liposome. The transition metal-based loading technique utilizes transition metals to drive the uptake of the agents into liposomes via complexation or coordination. Overall, a much higher encapsulation efficiency of the therapeutic agent can be achieved (e.g., >90%) by using the active loading technique as compared to that obtained from the passive loading technique.

The term “mean particle size” refers to the average diameter of the liposome. This can be measured by instrument based on dynamic light scattering.

The term “substituted ammonium” means that the hydrogen atoms in the ammonium ion are substituted with one or more alkyl group or some other organic group to form a substituted ammonium ion.

The term “triple negative breast cancer” refers to a type of breast cancer from which s the cancer cells do not have estrogen or progesterone receptors, and also do not make enough of the protein called human epidermal growth factor receptor 2 (HER2). Namely, the cells test “negative” on all three tests of the above receptors.

The term “non-small cell lung cancer” (NSCLC) refers to any type of epithelial lung cancer other than small cell lung cancer (SCLC). The most common types of NSCLC are squamous cell carcinoma, large cell carcinoma, and adenocarcinoma, but there are several other types that occur less frequently, and all types can occur in unusual histologic variants. %

The term “renal cell cancer” (RCC) refers to a type of kidney cancer that originates in the lining of the proximal convoluted tubule, a part of the very small tubes in the kidney that transport primary urine. RCC is the most common type of kidney cancer in adults, responsible for approximately 90-95% of cases.

The term “drug-resistant cancer” refers to the type of cancer that show resistance to the given therapeutic agents. Drug resistance occurs when cancer cells don't respond to a drug that is usually able to kill or weaken them. Drug resistance may be present before treatment is given (intrinsic resistance) or may occur during or after treatment with the drug (acquired resistance). In cancer treatment, there are many things that may cause resistance to anticancer drugs. For example, DNA changes or other genetic changes may change the way the drug gets into the cancer cells or the way the drug is broken down within the cancer cells. Drug resistance can lead to cancer treatment not working or to the cancer coming back.

The following abbreviations are used in this application:
AE: Adverse event;
AFA: Afatinib;
API(s): Active pharmaceutical ingredient(s);
CAR: Carfilzomib;
CD: Cyclodextrin;
CER: Ceritinib;
Chol: Cholesterol;
CI: Combination index;
DAS: Dasatinib;
DDPC: 1,2-Didecanoyl-sn-glycero-3-phosphocholine;
DEPC: 1,2-Dierucoyl-sn-glycero-3-phosphocholine;
DLPC: 1,2-dilauroyl-sn-glycero-3-phosphocholine;
DMPC: 1,2-dimyristoyl-sn-glycero-3-phosphocholine;
DPPC: 1,2-dipalmitoyl-sn-glycero-3-phosphocholine;
DMPG: 1,2-Dimyristoyl-sn-glycero-3-phosphoglycerol;
DPPG: 1,2-Dipalmitoyl-sn-glycero-3-phosphoglycerol;
DSPC: 1,2-distearoyl-sn-glycero-3-phosphocholine;
DSPG: 1,2-Distearoyl-sn-glycero-3-phosphoglycerol;
DOX: doxorubicin;
EDTA: Ethylenediaminetetraacetic acid;
ED₇₅ and ED₉₀: Effective dose required to affect 75 and 90% of the cells in cell culture;
Fa: Fraction affected;
GIST: Gastrointestinal stromal tumor;
HBS: HEPES buffered saline (20 mM HEPES, 150 mM NaCl, pH 7.4):
HEPES: N-2-hydroxylethyl-piperazine-N-2-ethanesulfonic acid;
HSPC: L-α-phosphatidylcholine, hydrogenated;
HP-β-CD: Hydroxypropyl-β-cyclodextrin;
LUV: Large unilamellar vesicle;
MLV: Multilamellar vesicle;
mPEG-2000-DSPE, sodium salt: N-(Carbonyl-methoxypolyethylenglycol-2000)-1,2-distearoyl-sn-glycero-3-phosphoethanolamine sodium, with polyethylene glycol of average molecular weight 2000;
MTT: 3-(4,5-dimethylthiazol-2-yl)-2,5-diphenyl-2-H tetrazolium bromide;
NSCLC: Non-small cell lung cancer;
PEG-2000-DMG: 1-monomethoxypolyethyleneglycol-2,3-dimyristylglycerol with
polyethylene glycol of average molecular weight 2000;
PG: Phosphatidylglycerol;
PSPC: 1-palmitoyl-2-stearoyl-sn-glycero-3-phosphocholine;
RCC: Renal cell carcinoma;
SBE-α-CD: Sulfobutylether-a-cyclodextrin;
SBE-β-CD: Sulfobutylether-β-cyclodextrin:
SBE-γ-CD: Sulfobutylether-γ-cyclodextrin;
SMPC: 1-stearoyl-2-myristoyl-sn-glycero-3-phosphocholine;
SOPC: 1-stearoyl-2-oleoyl-sn-glycero-3-phosphocholine;
SOS: Sucrose octasulfate;
SPPC: 1-stearoyl-2-palmitoyl-sn-glycero-3-phosphocholine;
SUV: Small unilamellar vesicle;
TEA: Triethylamine;
TEA-phytate: Triethylammonium salt of phytate;
TEA-SOS: Triethylammonium salt of sucrose octasulfate;
TEA-SBE-β-CD: Triethylammonium salt of sulfobutylether-β-cyclodextrin;
Tris: Tris(hydroxymethyl) aminomethane;
Tris-SBE-β-CD: Tris(hydroxymethyl) aminomethane salt of sulfobutylether-β-cyclodextrin;
Tris-SOS: Tris(hydroxymethyl) aminomethane salt of sucrose octasulfate.

### EXAMPLES

The following examples are provided to illustrate, but not to limit, the invention disclosed.

### Example 1

### Preparation of Trapping Agents Based on Polyanions

Trapping agent involved in active drug loading in liposome plays a critical role on payload encapsulation, retention as well as its dissolution profiles. The preparation on the such polyanion-based trapping agents is described in detail below.

### Preparation of Ammonium or Substituted Ammonium Salt of SOS and SBE-β-CD:

An ion exchange column was first packed with sulfonated polystyrene-divinylbenzene copolymer-based cation exchange resin beads. Then, the resin was equilibrated with ˜1N HCl, and subsequently washed with deionized water until the pH of the eluate was close to neutral. After that, solution of the sodium salt of sucrose octasulfate (SOS) or SBE-β-CD was added to the column and eluted with deionized water. The eluate containing the hydrogen form of SOS or SBE-β-CD was then titrated with triethylamine to a pH of 4.0-7.0. The resulting polyanion salts were named as triethylammonium SOS (TEA-SOS) or triethylammonium SBE-β-CD (TEA-SBE-β-CD), respectively. In other cases, ammonium hydroxide or tris(hydroxymethyl) aminomethane (Tris) or triethanolamine (TEOA) was used as the base for the titration of the hydrogen form of SOS or SBE-β-CD to generate the corresponding salt of the polyanion.

### Example 2

### General Protocol on the Preparation of Drug-Loaded Liposomes for Poorly-Water Soluble Compounds Through Active Loading

General steps to prepare the drug loaded liposome through active loading are the following: (1) Lipid hydration and particle size reduction (2) Dialysis (3) Active drug loading and finally (4) Free drug removal.

Detailed procedure is described as follows: A lipid mixture of DSPC, cholesterol and PEGylated lipid (e.g., mPEG-DSPE) and optionally DSPG are dissolved in ethanol and added into an aqueous solution containing one of the following trapping agents: ammonium sulfate, TEA-SOS, TEA-SBE-β-CD, or TEA-phytate at 50-70° C. Optionally, a buffering agent (e.g., HEPES) can be included within the above aqueous solution. Subsequently, the organic phase and the aqueous phase are mixed under vigorous stirring for approximately 30 minutes to allow for multilamellar vesicles (MLVs) formation. The liposome suspension is then subjected to size reduction (e.g., extrusion) through 100 nm and/or 50 nm polycarbonate membranes at elevated temperature (50-70° C.) to obtain the desired liposome particle size (70-120 nm) and particle size distribution (PDI<0.2), and then the liposome is quickly chilled to room temperature. After that, the external trapping agent outside the liposome is removed by a dialysis process against deionized water. Unloaded liposomes containing a trapping agent and optionally a buffering agent inside the vesicle are prepared based on the above process.

To load the drug into the liposome, drug solution is firstly prepared before mixing with the liposome. To this end, a poorly water-soluble compound (e.g., carfilzomib) is added and dissolved into an aqueous solution containing a buffering agent (e.g., citric acid) at pH 2.0-6.0 and a solubility improving agent (e.g., sodium salt of SBE-β-CD). In some cases, two or more drug compounds are dissolved in the buffer solution at this step to aim for combined drug loadings. Subsequently, the drug solution is mixed with the unloaded liposomes prepared above, and the drug loading is allowed to proceed at 50-70° C. for one hour. This active drug loading approach generally results in a drug encapsulation efficiency higher than 90%. Unloaded drug and external solubility improving agent can be removed from the liposome by one of the following methods: dialysis, size-exclusion chromatography or ultracentrifugation, or other suitable methods. An encapsulation efficiency is generally higher than 99% after the free drug removal step. Finally, liposome drug product is aseptically filtered and filled into Type I borosilicate glass vials for storage at 2-8° C. condition.

### Example 3

### General Protocols on the Physicochemical Characterization of the Liposomes

### Particle Size and Zeta-Potential.

Hydrodynamic particle size, polydispersity index (PDI) and the zeta-potential (i.e., particle surface charge) of the liposome drug product are measured using Nano-S90 ZetaSizer (Malvern Instruments, UK). Each sample is adequately diluted with distilled water prior to measurement.

### Morphological Characterization.

Cryo-Transmission Electron Microscopy (Cryo-TEM) is employed to examine the size and morphology of the liposome using a Cryogenic TEM-Titan Krios 80/300 Key transmission electron microscope (ThermoFisher Scientific).

### Drug Loading and Encapsulation.

Drug content (Assay) of the liposome product was determined by dissolving a known quantity of loaded liposome in Triton-X100 aqueous solution and the drug content is then quantified by HPLC-UV analysis. The free drug content was determined by first separating the free drug from the liposome through size exclusion chromatography (SEC), and then the unloaded drug content in the corresponding fraction is quantified by HPLC-UV analysis. The drug encapsulation efficiency (EE %) is calculated as the free drug content subtracted from the total drug content divided by the total drug content.

### In-Vitro Drug Release Study.

In-vitro release of drug loaded liposomes can be evaluated through a dialysis-based method. For example, a defined volume of liposome product (˜1-2 mL) is first added into a dialysis bag (molecular weight cutoff 10 kDa), which is pre-hydrated overnight in phosphate-buffered saline (PBS) buffer at pH 7.4. The dialysis bag was then placed into a glass reservoir containing 150 mL PBS (pH 7.4). The dissolution study is conducted at 37° C. under gentle stirring. Aliquots (˜1 mL) of the release media are sampled at predetermined time intervals and the reservoir is replenished with equal volumes of fresh media. The drug content of the specific compound is determined by HPLC-UV method. Cumulative drug release profile is then generated based on the released drug content at each time point.

### Example 4

### Preparation of Carfilzomib Loaded Liposome Through Active Loading

The detailed drug-loaded liposome preparation procedure was described in Example 2. Herein, four different types of trapping agents were used (Table 1), namely, (1) TEA-SOS (2) TEA-SBE-β-CD (3) TEA-phytate and (4) ammonium sulfate. By varying the concentration of trapping agents, different sulfate or sulfo or phosphate to API molar ratios were applied for each trapping agent studied (Table 1). All liposome drug product in Table 1 contains the following composition: (1) lipid composition: DSPC (59 mol %), cholesterol (40 mol %) and mPEG-DSPE (1 mol %); (2) 100 mg/ml of sodium salt of SBE-β-CD was used as the solubility improving agent to solubilize carfilzomib in all drug loadings and (3) 10 mM citric acid at pH 6.50. The external medium pH was at 3.50 during drug loading. In general, the higher the molar ratio of sulfate or sulfo or phosphate to API, the higher the drug encapsulation efficiency can be obtained. Another conclusion from Table 1 is that the use of polyanions as trapping agents gave significantly higher encapsulation efficiency (>60%) as compared to that of ammonium sulfate (<30%).

TABLE 1
Physicochemical properties of carfilzomib loaded liposomes using
sodium salt of SBE-β-CD as the solubility enhancement agent
with different types of trapping agents. All liposomes contain
the following lipid composition: DSPC, cholesterol and mPEG-DSPE.

| | | | Drug to | | |
|---|---|---|---|---|---|
| | | Sulfate, sulfo or | lipid | | |
| | | phosphate to | weight | | Encapsulation |
| | | API molar ratio | ratio in | Particle size | efficiency |
| Lot# | Trapping agent | in feed | feed | (nm)/PDI | (EE %) |
| 0407-1 | TEA-SOS | 1.9 | 1:8 | 100/0.052 | 75% |
| 0407-2 | | 3.7 | 1:8 | 107/0.069 | 91% |
| 0506 | | 7.3 | 1:16 | 103/0.074 | 92% |
| 0413 | TEA-SBE-β-CD | 1.9 | 1:8 | 100/0.050 | 67% |
| 0525-1 | | 3.7 | 1:8 | 103/0.050 | 77% |
| 0406 | TEA-Phytate | 1.9 | 1:8 | 99/0.050 | 61% |
| 0525-2 | | 3.7 | 1:8 | 112/0.048 | 74% |
| 0407-3 | (NH₄)₂SO₄ | 1.7 | 1:8 | 88/0.105 | 29% |

In some cases, DSPG is also included in the lipid composition with the aim to improve the physical stability of the liposome. In the example shown in Table 2, the lipid composition is the following: DSPC (58.5 mol %), cholesterol (39.5 mol %), mPEG-DSPE (1 mol %) and DSPG (1 mol %). The final formulation contains the following: (1) 100 mg/ml of sodium salt of SBE-β-CD was used as the solubility enhancement agent to solubilize carfilzomib during drug loadings and (2) 10 mM citric acid at pH 6.50. The external medium pH was at 4.0 during drug loading. A free drug removal process using dialysis was employed here to remove the unloaded carfilzomib which resulted in a >99% encapsulation efficiency in the final drug product.

TABLE 2
Physicochemical properties of carfilzomib loaded liposomes. Free
drug removal process applied. All liposomes contain the following
lipid composition: DSPC, cholesterol, mPEG-DSPE and DSPG.

| | | Sulfate to | Drug to | Particle | EE % before | EE % after |
|---|---|---|---|---|---|---|
| | Trapping | API molar | lipid weight | size (nm)/ | unloaded | unloaded |
| Lot# | agent | ratio in feed | ratio in feed | PDI | drug removal | drug removal |
| 0707 | TEA-SOS | 2.9 | 1:4 | 103/0.037 | 95% | >99% |

### Example 5

### Effect of Drug Loading pH on Encapsulation Efficiency of Carfilzomib

The detailed drug-loaded liposome preparation procedure was described in Example 2. Sodium salt of SBE-β-CD was used as the solubility improving agent. A total of six drug loading pH was studied (Table 3), i.e., pH 5.0, pH 4.75, pH 4.50, pH 4.0, pH 3.50 and pH 3.0. All final liposome drug product contains the following composition: (1) lipid composition: DSPC (59 mol %), cholesterol (40 mol %) and mPEG-DSPE (1 mol %) (2) 100 mg/ml of sodium salt of SBE-β-CD and (3) a buffering agent at pH 6.50. As shown in Table 3, the results reflect that the highest encapsulation efficiency was achieved when the drug loading pH was used as 4.0. Drug loading pH deviated from the value above resulted in decreased drug encapsulation efficiency.

TABLE 3
Effect of drug loading pH on EE %. All liposomes contain
the following lipid composition: DSPC, cholesterol and
mPEG-DSPE. TEA-SOS was used as the trapping agent.

| | Sulfate to API | Drug to lipid | Drug | | Encapsulation |
|---|---|---|---|---|---|
| | molar ratio in | weight ratio | loading | Particle size | efficiency |
| Lot# | feed | in feed | pH | (nm)/PDI | (EE %) |
| 0901 | 1.8:1 | 1:4 | 5.0 | 99/0.041 | 82% |
| 0912-1 | 1.8:1 | 1:4 | 4.75 | 100/0.056 | 93% |
| 0912-2 | 1.8:1 | 1:4 | 4.50 | 99/0.054 | 94% |
| 0817-1 | 1.8:1 | 1:4 | 4.0 | 98/0.053 | 97% |
| 0817-2 | 1.8:1 | 1:4 | 3.50 | 98/0.042 | 96% |
| 0817-3 | 1.8:1 | 1:4 | 3.0 | 97/0.052 | 90% |

### Example 6

### Drug Loading Capacity Study of Carfilzomib in Liposome

The detailed drug-loaded liposome preparation procedure was described in Example 2. Sodium salt of SBE-β-CD was used as the solubility improving agent. Different drug to lipid weight ratio in feed was explored to study the drug loading capacity of the liposome. All final liposome drug product contains the following composition: (1) lipid composition: DSPC (59 mol %), cholesterol (40 mol %) and mPEG-DSPE (1 mol %) (2) 100 mg/ml of sodium salt of SBE-β-CD and (3) a buffering agent pH 6.50. The external medium pH was 4.0 when drug loading was performed. As shown from Table 4 below, drug loading content as high as close to 30 wt % within the liposome can be achieved.

TABLE 4
Drug loading capacity study. High drug loading content of carfilzomib
in liposome can be achieved. TEA-SOS was used as the trapping agent.

| | Sulfate to | Drug to lipid | | Encapsulation | |
|---|---|---|---|---|---|
| | API molar | weight ratio | Particle size | efficiency | Drug loading |
| Lot# | ratio in feed | in feed | (nm)/PDI | (EE %) | content (wt %) |
| 0817-1 | 1.8:1 | 1:4 | 98/0.053 | 97% | 20 wt % |
| 0912-3 | 1.1:1 | 1:2.3 | 97/0.025 | 94% | 29 wt % |

### Example 7

### Preparation of Dasatinib Loaded Liposome Through Active Loading

The detailed drug-loaded liposome preparation procedure was described in Example 2. Sodium salt of SBE-β-CD was used as the solubility improving agent. Different trapping agents were used for dasatinib encapsulation, and the product characterization results are shown in Table 5. All final liposome drug product contains the following composition: (1) lipid composition: DSPC (58.5 mol %), cholesterol (39.5 mol %), mPEG-DSPE (1 mol %) and DSPG (1 mol %) (2) 100 mg/ml of sodium salt of SBE-β-CD and (3) 10 mM citric acid at pH 6.50. The external medium pH was at 3.50 during drug loading. As shown from the results, the trapping agent of TEA-SOS gives the highest dasatinib encapsulation efficiency.

TABLE 5
Physicochemical properties of dasatinib loaded liposomes. All liposomes contain
the following lipid composition: DSPC, cholesterol, mPEG-DSPE and DSPG.

| | | Sulfate, sulfo | | | |
|---|---|---|---|---|---|
| | | or phosphate to | Drug to lipid | | Encapsulation |
| | | API molar | weight ratio | Particle size | efficiency |
| Lot# | Trapping agent | ratio in feed | in feed | (nm)/PDI | (EE %) |
| 0506 | TEA-SOS | 2.6 | 1:8 | 100/0.063 | 94% |
| 0525-1 | TEA-SBE-β-CD | 2.6 | 1:8 | 101/0.028 | 73% |
| 0525-2 | TEA-Phytate | 2.6 | 1:8 | 104/0.051 | 76% |

### Example 8

### Preparation of Ceritinib Loaded Liposome Through Active Loading

The detailed drug-loaded liposome preparation procedure was described in Example 2. Sodium salt of SBE-β-CD was used as the solubility improving agent. Different trapping agents were used for ceritinib encapsulation, and the product characterization results are shown in Table 6. All final liposome drug product contains the following composition: (1) lipid composition: DSPC (58.5 mol %), cholesterol (39.5 mol %), mPEG-DSPE (1 mol %) and DSPG (1 mol %) (2) 100 mg/ml of sodium salt of SBE-β-CD and (3) a buffering agent pH 6.50. The external medium pH was at 3.50 during drug loading. As shown from the results, all three trapping agents give close to 100% encapsulation efficiency of ceritinib.

TABLE 6
Physicochemical properties of ceritinib loaded liposomes. All liposomes contain
the following lipid composition: DSPC, cholesterol, mPEG-DSPE and DSPG.

| | | Sulfate, sulfo | | | |
|---|---|---|---|---|---|
| | | or phosphate to | Drug to lipid | | Encapsulation |
| | | API molar | weight ratio | Particle size | efficiency |
| Lot# | Trapping agent | ratio in feed | in feed | (nm)/PDI | (EE %) |
| 0506 | TEA-SOS | 2.9 | 1:8 | 102/0.077 | 99% |
| 0525-1 | TEA-SBE-β-CD | 2.9 | 1:8 | 100/0.041 | 97% |
| 0525-2 | TEA-Phytate | 2.9 | 1:8 | 107/0.046 | 98% |

### Example 9

### In-Vitro Cytotoxicity Study for Drug Synergy Determination

For combined drug regimens, the two or more compounds involved in the combination can exhibit synergistic, additive, or antagonistic interactions depending on the molar drug ratios. To study those drug-drug interactions in a quantitative approach, a combination index (CI)-based method was used in this study following previously reported procedure (Chou, T. C., J. Theor. Biol. (1976) 39:253-276).

The general protocol on cell culture and liposome drug efficacy evaluation are briefly stated as follows. Adherent cancer cell lines are collected in their logarithmic growth phase using standard cell culture techniques. Cell concentrations are determined by hemocytometer and then diluted by their respective media to the targeted cell concentrations. Cells are then seeded onto a 96-well plate. The map on the plate is designed to include treatment groups, cell-only control (no drug treatment) and media-only control (no cell and no drug treatment). Cell seeding concentrations are optimized such that 48 hours after cell plating a MTT assay performed on the untreated control cells would generate an absorbance value of around 1.0 at 590 nm.

The cell seeded plate is incubated for 24 hours at 37° C. and 5% CO₂ in a standard cell culture incubator before drug treatment. The following day, drug dilutions on either solo drug or drug combinations at defined molar drug ratios are prepared using respective cell culture media. The cell culture media in the 96-well plate is then replaced by fresh media containing the drug or drug combinations. After another 24 hours of incubation, cell viability is assessed by the MTT assay following the manufacture's protocols. Relative percent survival is determined by subtracting absorbance values obtained by media-only wells from drug treated wells and then normalizing to the no-drug control wells (cell only control). The fraction of cells affected (fa), or cell growth inhibition (%) at each drug concentration is subsequently calculated for each well. The effect of drug combinations is then calculated and processed by a software named “CompuSyn” for drug synergy analysis. The program employs the median-effect analysis algorithm, which produces the Combination Index value as a quantitative indicator of the degree of synergy. Based on this analysis method, a CI<0.9 indicates synergy, the range 0.9≤CI<1.1 reflects additive effect and a CI>1.1 indicates antagonism. CI plots are typically illustrated with CI representing the y-axis versus the proportion of cells affected, or fraction affected (Fa), on the x-axis. The synergistic ratio of drug combinations is identified and then used for future studies.

### Example 10

### In-Vitro Evaluation on Afatinib and Dasatinib Combination for Synergy in Cancer Cells

To identify the molar ratios of afatinib and dasatinib (AFA/DAS) that are synergistic, various drug-to-drug ratios were tested for their cytotoxic effects in cancer cell lines in vitro. Measurement of the cytotoxic effects was performed using AFA/DAS at 5:1, 2.5:1, 1:1, 1:2.5 and 1:5 molar ratios in HCC827 non-small cell lung cancer cell line. Cytotoxic effect from the treatment of AFA alone and DAS alone in the corresponding cell line were included as controls.

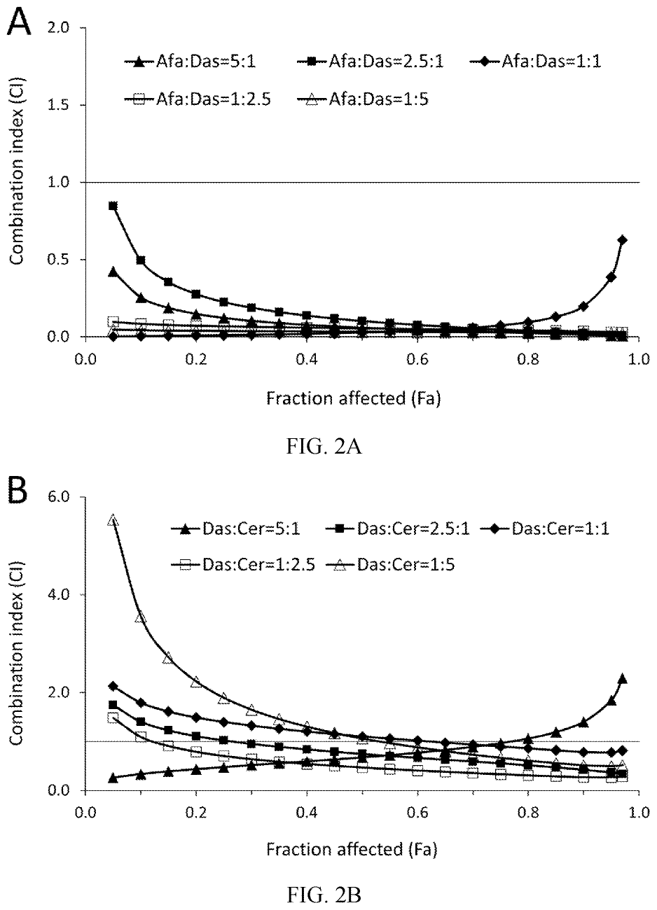

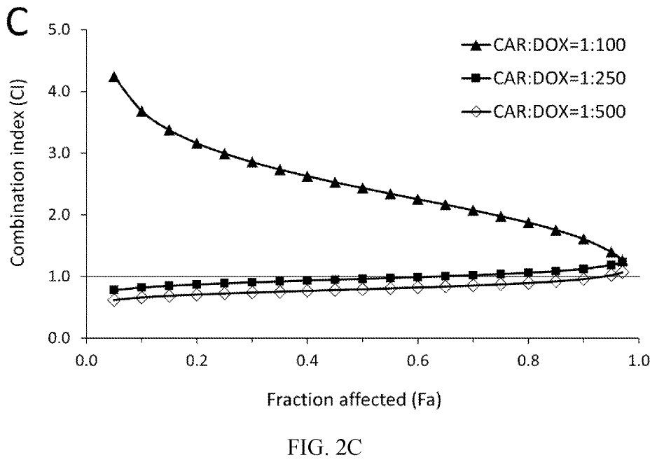

The detailed procedures for cell culture, drug treatment, MTT assay for cytotoxicity measurement and CI value calculation were described in the Example 9. FIG. 2A shows the representative plot based on CI values as a function of cell growth inhibition (i.e., cell fraction affected, Fa) at different AFA/DAS ratios evaluated in HCC827 cell lines. It was found that all above tested drug molar ratios give synergistic tumor cell growth inhibition effect.

### Example 11

### In-Vitro Evaluation on Dasatinib and Ceritinib Combination for Synergy in Cancer Cells

To identify the molar ratios of dasatinib/ceritinib (DAS/CER) that are synergistic, various drug-to-drug ratios were tested for their cytotoxic effects in cancer cell lines in vitro. Measurement of the cytotoxic effects was performed using DAS/CER at 5:1, 2.5:1, 1:1, 1:2.5 and 1:5 molar ratios in HCC827 non-small cell lung cancer cell line. Cytotoxic effect from the treatment of DAS alone and CER alone in the corresponding cell line were included as controls.

The detailed procedures for cell culture, drug treatment, MTT assay for cytotoxicity measurement and CI value calculation were described in the Example 9. FIG. 2B shows the representative plot based on CI values as a function of cell growth inhibition (i.e., cell fraction affected, Fa) at different DAS/CER ratios evaluated in HCC827 cell lines. It was found that at the following drug molar ratios, synergistic tumor cell growth inhibition effect was obtained: DAS:CER=2.5:1, 1:1 and 1:2.5.

### Example 12

### In-Vitro Evaluation on Carfilzomib and Doxorubicin Combination for Synergy in Cancer Cells

To identify the molar ratios of carfilzomib and doxorubicin (CAR/DOX) that are synergistic, various drug-to-drug ratios of CAR/DOX were tested for their cytotoxic effects in cancer cell lines in vitro. Measurement of the cytotoxic effects was performed using CAR/DOX at 1:100, 1:250 and 1:500 molar ratios in H929 myeloma cell line. Cytotoxic effect from the treatment of CAR alone and DOX alone in the corresponding cell line were included as controls.

The detailed procedures for cell culture, drug treatment, MTT assay for cytotoxicity measurement and CI value calculation were described in the Example 9. FIG. 2C shows the representative plot based on CI values as a function of cell growth inhibition (i.e., cell fraction affected, Fa) at different CAR/DOX ratios evaluated in H929 cell lines. It was found that at the CAR/DOX molar ratio of 1:500, synergistic tumor cell growth inhibition effect was observed.

### Example 13

### Preparation of Carfilzomib and Doxorubicin Co-Loaded Liposomes

The detailed drug-loaded liposome preparation procedure was described in Example 2. Sodium salt of SBE-β-CD was used as the solubility improving agent. During the drug loading step, both the carfilzomib and the doxorubicin at defined molar ratio as shown in Table 7 were dissolved in the citric acid buffer solution containing 100 mg/ml sodium salt of SBE-β-CD, then the drug solution was mixed with the liposome suspension and the loading proceeded as described in Example 2. In this example, doxorubicin is water soluble, and carfilzomib is poorly water soluble. The external medium pH was at 3.50 during drug loading. TEA-SOS was used as the trapping agents for this combo drug encapsulation, and the product characterization results are shown in Table 7. The final liposome drug product contains the following composition: (1) lipids: DSPC (58.5 mol %), cholesterol (39.5 mol %), mPEG-DSPE (1 mol %) and DSPG (1 mol %) (2) 100 mg/ml of sodium salt of SBE-β-CD and (3) 10 mM citric acid at pH 6.50. High drug encapsulation efficiency for both of the compounds were achieved.

TABLE 7
Physicochemical properties of carfilzomib (CAR) and doxorubicin
(DOX) co-loaded liposomes with the lipid composition of
the following: DSPC, cholesterol, mPEG-DSPE and DSPG.

| | | | | Drug to | | |
|---|---|---|---|---|---|---|
| | | | | lipid | | |
| | | Sulfate to | Molar | weight | Particle | Encapsulation |
| | Trapping | API molar | drug ratio | ratio in | size (nm)/ | efficiency |
| Lot# | agent | ratio in feed | CAR:DOX | feed | PDI | (EE %) |
| 0518 | TEA-SOS | 3.4 | 1:1 | 1:8 | 103/0.042 | 99% (DOX) |
| | | | | | | 95% (CAR) |

### Example 14

### Preparation of Dasatinib and Ceritinib Co-Loaded Liposomes

The detailed drug-loaded liposome preparation procedure was described in Example 2. Sodium salt of SBE-β-CD was used as the solubility improving agent. During the drug loading step, both the dasatinib and the ceritinib at defined molar ratio as shown in Table 8 were dissolved in the citric acid buffer solution containing 100 mg/ml sodium salt of SBE-β-CD, then the drug solution was mixed with the liposome suspension and the loading proceeded as described in Example 2. The external medium pH was at 3.50 during drug loading. In this example, both of the compound are poorly water soluble. Different trapping agents (i.e., TEA-SOS, TEA-SBE-β-CD and TEA-Phytate) were used for this combo drug encapsulation, and the product characterization results are shown in Table 8. The final liposome drug product contains the following composition: (1) DSPC (58.5 mol %), cholesterol (39.5 mol %), mPEG-DSPE (1 mol %) and DSPG (1 mol %) (2) 100 mg/ml of sodium salt of SBE-β-CD and (3) a buffering agent at pH 6.50. TEA-SOS trapping agent gives high drug encapsulation efficiency for both of the compounds.

TABLE 8
Physicochemical properties of dasatinib (DAS) and ceritinib
(CER) co-loaded liposomes with the lipid composition of
the following: DSPC, cholesterol, mPEG-DSPE and DSPG.

| | | | | Drug to | | |
|---|---|---|---|---|---|---|
| | | Sulfate, sulfo | | lipid | | |
| | | or phosphate | Molar | weight | Particle | Encapsulation |
| | | to API molar | drug ratio | ratio in | size (nm)/ | efficiency |
| Lot# | Trapping agent | ratio in feed | DAS:CER | feed | PDI | (EE %) |
| 0518 | TEA-SOS | 2.8 | 1:1 | 1:8 | 99/0.047 | 91% (DAS) |
| | | | | | | 99% (CER) |
| 0525-1 | TEA-SBE-β-CD | 2.8 | 1:1 | 1:8 | 101/0.046 | 72% (DAS) |
| | | | | | | 98% (CER) |
| 0525-2 | TEA-Phytate | 2.8 | 1:1 | 1:8 | 106/0.039 | 67% (DAS) |
| | | | | | | 99% (CER) |

### Example 15

### Preparation of Afatinib and Dasatinib Co-Loaded Liposomes

The detailed drug-loaded liposome preparation procedure was described in Example 2. Sodium salt of SBE-β-CD was used as the solubility improving agent. During the drug loading step, both the afatinib and the dasatinib at defined molar ratio as shown in Table 9 were dissolved in the citric acid buffer solution containing 100 mg/ml sodium salt of SBE-β-CD, then the drug solution was mixed with the liposome suspension and the loading proceeded as described in Example 2. In this example, afatinib is water-soluble and dasatinib is poorly water soluble. The external medium pH was at 4.0 during drug loading. TEA-SOS was used as the trapping agents for this combo drug encapsulation, and the product characterization results are shown in Table 9. The final liposome drug product contains the following composition: (1) DSPC (58.5 mol %), cholesterol (39.5 mol %), mPEG-DSPE (1 mol %) and DSPG (1 mol %) (2) 100 mg/ml of sodium salt of SBE-β-CD and (3) a buffering agent at pH 6.50. High drug encapsulation efficiency for both of the compounds were achieved.

TABLE 9
Physicochemical properties of afatinib (AFA) and dasatinib
(DAS) co-loaded liposomes with the lipid composition of
the following: DSPC, cholesterol, mPEG-DSPE and DSPG.

| | | Sulfate to API | Molar | Drug to lipid | Particle | Encapsulation |
|---|---|---|---|---|---|---|
| | Trapping | molar ratio in | drug ratio | weight ratio | size (nm)/ | efficiency |
| Lot# | agent | feed | AFA:DAS | in feed | PDI | (EE %) |
| 0627 | TEA-SOS | 4.9 | 1:1 | 1:8 | 106/0.053 | 98% (AFA) |
| | | | | | | 92% (DAS) |

### Example 16

Use of HP-β-CD as solubility improving agent for remote loading of carfilzomib The detailed drug-loaded liposome preparation procedure was described in Example 2. In this example, hydroxypropyl-β-cyclodextrin (HP-β-CD) was used as the solubility improving agent and the result was compared to drug loading using SBE-β-CD for drug solubilization (Table 9). All liposomes in Table 10 contain the following composition: (1) DSPC (58.5 mol %), cholesterol (39.5 mol %), mPEG-DSPE (1 mol %) and DSPG (1 mol %); (2) 100 mg/ml of HP-β-CD or 100 mg/mL of sodium salt of SBE-β-CD and (3) a buffering agent at pH 6.50. The external medium pH was at 4.0 during drug loading. The results (Table 10) reflect that by using SBE-D-CD, a significantly higher EE % (i.e., 95%) can be obtained as compared to that from drug loading using HP-β-CD, which only gives an EE % of 57%. Also, formulation using SBE-β-CD exhibits narrow particle size distribution (i.e., 0.037) of the liposome. In contrast, formulation using HP-β-CD shows a very broad particle size distribution (i.e., 0.341) which indicates that HP-β-CD may cause liposome aggregation and negatively affect liposome stability and morphology.

TABLE 10
Effect of the type of solubility improving agent on carfilzomib (CAR) encapsulation.

| | | Solubility | Sulfate to | Drug to | Particle | Encapsulation |
|---|---|---|---|---|---|---|
| | Trapping | improving | API molar | lipid weight | size (nm)/ | efficiency |
| Lot# | agent | agent | ratio in feed | ratio in feed | PDI | (EE %) |
| 0722-2 | TEA-SOS | HP-β-CD | 2.9 | 1:4 | 164/0.341 | 57% |
| 0707 | TEA-SOS | SBE-β-CD | 2.9 | 1:4 | 103/0.037 | 95% |

### Example 17

### Loading of Carfilzomib at Controlled pH within the Intraliposomal Compartment

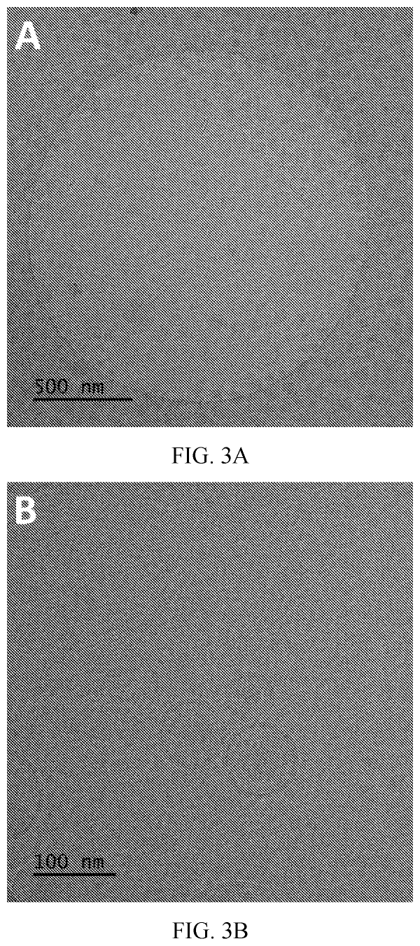

In this formulation, a buffering agent is included within the intraliposomal compartment with the aim to improve product stability. The detailed drug-loaded liposome preparation procedure was described in Example 2. Herein, 65 mM TEA-SOS was used as the trapping agent and the intraliposomal compartment also contains 25 mM HEPES buffer at the pH of 7.0. The lipid composition contains the following: DSPC (59 mol %), cholesterol (40 mol %) and mPEG-DSPE (1 mol %). For drug loading, 100 mg/ml of sodium salt of SBE-β-CD and 10 mM citric acid was used to solubilize carfilzomib. After the drug loading step, an encapsulation efficiency of 93% was obtained. Then, a free drug removal process was applied by dialyzing the product against a solution of HEPES buffer at pH 7.0 plus 0.9% NaCl. After the free drug removal step, the drug encapsulation efficiency of the final product was increased to 99.2%. The final drug product contains 1 mg/mL carfilzomib and 4 mg/mL total lipid and exhibited an average particle size of 98.4 nm with a polydispersity of 0.044 analyzed by Zetasizer based on dynamic light scattering. Morphological characterization of the carfilzomib liposome was performed by using cryogenic transmission electron microscopy (cryo-TEM). And the representative images are shown in FIG. 3A and FIG. 3B. The dark area inside the core of the liposome reflects the encapsulation of the drug compound.

### Example 18

### Impact of Intraliposomal pH on Carfilzomib Stability

Carfilzomib-loaded liposome with an aqueous core contains 65 mM TEA-SOS at pH 5.20 was prepared according to the procedure described in Example 2. Carfilzomib-loaded liposome with an aqueous core contains 65 mM TEA-SOS and 25 mM HEPES at pH 7.0 was prepared based on the procedure described in Example 17. All other formulation composition and process conditions are the same between the above two formulations. In both cases, the lipid composition contains the following: DSPC (59 mol %), cholesterol (40 mol %) and mPEG-DSPE (1 mol %). Also, sodium salt of SBE-β-CD was used as the solubility improving agent for drug loadings. As shown in Table 11, at an internal pH of 5.20, a total of 1.8% drug-related impurities was obtained in the final drug product. In contrast, no drug related impurities were found in the product at an intraliposomal pH of 7.0.

TABLE 11
Impact of the pH of liposome aqueous interior core on carfilzomib stability

| | Sulfate to | Drug to | Liposome aqueous | |
|---|---|---|---|---|
| | API molar | lipid weight | interior core | Carfilzomib related |
| Lot # | ratio in feed | ratio in feed | composition | impurities in final product |
| 0920 | 2.9 | 1:4 | 65 mM TEA-SOS at | 1.8% |
| | | | pH 5.20 | |
| 0829 | 2.9 | 1:4 | 65 mM TEA-SOS and | Not detectable (Below |
| | | | 25 mM HEPES at | detection limit) |
| | | | pH 7.0 | |

### Example 19

### In Vivo Pharmacokinetics (PK) Study in Mice of Carfilzomib-Loaded Liposome

The carfilzomib-loaded liposome was prepared based on the description in Example 17. In-vivo pharmacokinetics study was carried out in BALB/c nude mice. Carfilzomib drug solution (free drug) and liposomal carfilzomib were administered intravenously via the tail vein into the mice and the plasma concentration of carfilzomib of both formulations were monitored over time by LC-MS. The injection dose of both drug formulations was 5.0 mg/kg. After intravenous administration, blood was collected at pre-determined time points (3 mice per time point) and was placed into EDTA coated micro containers. The samples were then centrifuged to separate plasma. Plasma levels of carfilzomib were quantified by using LC-MS. PK parameters were then calculated from measured drug plasma concentrations over time.

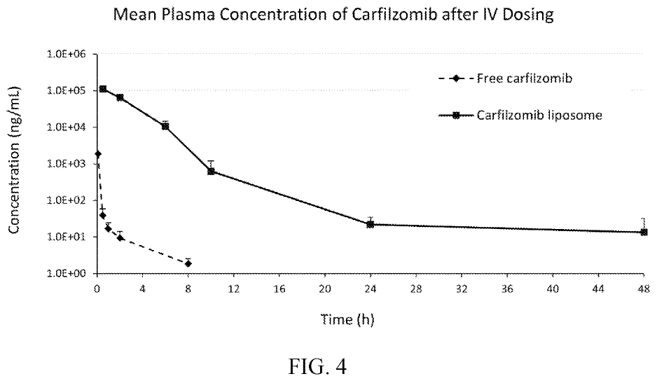

PK results are shown in the table below as well as in FIG. 4. Liposomal carfilzomib exhibited a 10-fold increase of in vivo half-life as compared to that from the free drug solution. For volume of distribution, a 152-fold reduction was obtained by the liposomal carfilzomib versus the free drug solution. Also, the former formulation also achieved a 665-fold increase of AUC as compared to the latter one. Overall, the liposome formulation of carfilzomib exhibited significantly prolonged blood circulation time when compared with the free drug solution.

TABLE 12
Key PK parameters of free carfilzomib
and liposomal carfilzomib in mice

| | Free | Liposomal |
|---|---|---|
| PK parameters | carfilzomib | carfilzomib |
| T_{1/2} (h) | 0.32 | 3.43 |
| Vdₛₛ (L/kg) | 5.36 | 0.0352 |
| Cl (mL/min/kg) | 170 | 0.266 |
| AUC₀₋ₗₐₛₜ (ng · h/mL) | 486 | 323,243 |

The foregoing embodiments and examples are provided for illustrative purposes only and are not intended to limit the scope of the invention. Many variations to those described above may be possible. Since various modifications and variations to the embodiments and examples described above will be apparent to those of skill in this art based on the present disclosure, such modifications and variations are within the spirit and scope of the present invention. All patent or non-patent literature cited are incorporated herein by reference in their entireties without admission of them as prior art.

1. Drug-loaded liposome particles comprising an interior core and an exterior lipid bilayer membrane, wherein the lipid bilayer membrane comprises an inner layer having an inner surface enclosing the interior core and an outer layer forming an outer surface of the liposome particle; wherein the interior core comprises an aqueous liquid medium and one or more active pharmaceutical ingredients encapsulated by the bilayer membrane, wherein at least one of the active pharmaceutical ingredients is poorly water-soluble; wherein the aqueous liquid medium of the interior core comprises a trapping agent and optionally a buffering agent; and wherein the drug-loaded liposome particles have a mean particle size between 10 nm and 450 nm, optionally between 25 nm and 300 nm or between 50 nm and 200 nm.

2. The drug-loaded liposome particles of claim 1, wherein the lipid bilayer membrane comprises:
a) a phospholipid selected from phosphatidylcholine (e.g., HSPC, DSPC, DPPC and DMPC), phosphatidylglycerol (e.g., DSPG, DPPG and DMPG), phosphatidylinositol, glycerol glycolipids, sphingoglycolipids (e.g., sphingomyelin), and combinations thereof, wherein the phospholipid is in an amount of at least 10 mol % of the total lipid present in the liposome particle;
b) cholesterol, or a derivative thereof, in an amount of from 5 mol % to 50 mol % of the total lipid present in the liposome particle; and
c) a conjugated lipid, which inhibits aggregation of liposomes, in an amount of from 0 mol % to 10 mol % of the total lipid present in the liposome particle.

3. The drug-loaded liposome particles of claim 2, wherein the conjugated lipid that inhibits aggregation of liposomes comprises a polyethyleneglycol (PEG)-lipid conjugate.

4. The drug-loaded liposome particles of claim 3, wherein the PEG has an average molecular weight in the range of about 1,500 Daltons to about 2,500 Daltons; and wherein optionally the PEG-lipid conjugate is mPEG2000-DSPE or PEG2000-DMG.

5. The drug-loaded liposome particles of claim 1, wherein the buffering agent is selected from acetic acid, citric acid, histidine, HEPES, lactic acid, succinic acid, phosphoric acid, tromethamine (Tris), and salts thereof, and combinations thereof.

6. The drug-loaded liposome particles of claim 1, wherein the liquid medium in the interior core comprises a trapping agent optionally selected from ammonium sulfate; ammonium or substituted ammonium salts of polyanionized sulfobutyl ether cyclodextrin; ammonium or substituted ammonium salts of polyanionized sulfated carbohydrates; ammonium or substituted ammonium salts of polyphosphate; metal salts; and combinations thereof.

7. The drug-loaded liposome particles of claim 6, wherein the ammonium salts of polyanionized sulfobutyl ether cyclodextrin are selected from TEA-SBE-α-cyclodextrin, TEA-SBE-β-cyclodextrin, TEA-SBE-γ-cyclodextrin, Tris-SBE-α-cyclodextrin, Tris-SBE-β-cyclodextrin and Tris-SBE-γ-cyclodextrin; the ammonium salts of polyanionized sulfated carbohydrates are selected from TEA-SOS and Tris-SOS; the ammonium salts of polyphosphate are selected from triethylammonium inositol hexaphosphate and tris(hydroxymethyl) aminomethane inositol hexaphosphate; and the metal salt is selected from calcium, copper, zinc, magnesium, manganese, nickel, or cobalt salts of acetate, carbonate, citrate, halide, sulfate, and gluconate.

8. The drug-loaded liposome particles of claim 1, wherein the active pharmaceutical ingredients are selected from afatinib, abemaciclib, abiraterone, acalabrutinib, alectinib, almonertinib, alpelisib, anlotinib, apatinib, avapritinib, axitinib, baricitinib, belinostat, binimetinib, bortezomib, bosutinib, brigatinib, bupivacaine, cabozantinib, capecitabine, carfilzomib, capmatinib, ceritinib, cobimetinib, copanslisib, crizotinib, dabrafenib, dacomitinib, dasatinib, delanzomib, docetaxel, doxorubicin, duvelisib, enasidenib, encorafenib, entrectinib, erdafitinib, erlotinib, everolimus, fedratinib, fostamatinib, fruquintinib, gefitinib, gemcitabine, gilteritinib, glasdegib, icotinib, ibrutinib, idarubicin, idelalisib, imatinib, ivosidenib, ixazomib, ixabepilone, lapatinib, larotrectinib, lenalidomide, lenvatinib, lorlatinib, marizomib, midostaurin, mitoxantrone, neratinib, netarsudil, nilotinib, nintedanib, niraparib, olaparib, oprozomib, osimertinib, paclitaxel, palbociclib, panobinostat, pazopanib, pemetrexed, pemigatinib, pexidartinib, ponatinib, pralsetinib, quizartinib, radotinib, regorafenib, ribociclib, ripretinib, rivastigmine, romidepsin, rucaparib, ruxolitinib, selpercatinib, selumetinib, sirolimus, sonidegib, sorafenib, sunitinib, talazoparib, tazemetostat, temsirolimus, tepotinib, tivozanib, tofacitinib, topotecan, trametinib, tucatinib, tucidinostat, upadacitinib, vandetanib, vemurafenib, venetoclax, vinorelbine, vismodegib, vorinostat, and zanubrutinib, and freebases, pharmaceutical salts, derivatives, and mixtures thereof.

9. The drug-loaded liposome particles of claim 1, wherein the active pharmaceutical ingredients are selected from:
a) carfilzomib encapsulated alone;
b) dasatinib encapsulated alone;
c) ceritinib encapsulated alone;
d) carfilzomib and doxorubicin co-encapsulated;
e) dasatinib and ceritinib co-encapsulated;
f) afatinib and dasatinib co-encapsulated;
g) carfilzomib and doxorubicin in about 1:50 to about 1:1000 molar ratio;
h) dasatinib and ceritinib in about 30:1 to about 1:30 molar ratio; and
i) afatinib and dasatinib in about 30:1 to about 1:30 molar ratio.

10. A pharmaceutical composition, comprising the drug-loaded liposome particles of claim 1 and a liposome dispersion liquid medium.

11. The pharmaceutical composition of claim 10, wherein the liposome dispersion liquid medium comprises water, a buffering agent, and a tonicity modifier; wherein optionally the buffering agent is selected from acetic acid, citric acid, histidine, HEPES, lactic acid, succinic acid, phosphate salt, tromethamine (Tris), and salts thereof; and wherein optionally the tonicity modifier is selected from sucrose, dextrose, mannitol, trehalose, and sodium chloride.

12. The pharmaceutical composition of claim 10, wherein two or more active pharmaceutical ingredients are co-encapsulated in the interior core of the liposome particles and can be released to function in a synergistic mode for efficacy, wherein the synergistic mode comprises that the active pharmaceutical ingredients can maintain a synergistic molar ratio in blood for at least one hour after administration of the pharmaceutical composition to a subject; and optionally wherein the synergistic molar ratio is a molar ratio such that when the ratio is provided to cancer cells relevant to the cancer in an in-vitro assay over a drug concentration range at which cell growth inhibition range is from about 0.20 to about 0.80 (i.e., the fraction of affected cells is in the range of about 20% to about 80%), a synergistic effect of at least 20% is exhibited within the cell growth inhibition range.

13. A method of treating a disease or condition in a subject in need of treatment with a therapeutic agent, the method comprising administering the subject a therapeutically effective amount of the drug-loaded liposome particles according to claim 1.

14. The method of claim 13, wherein the subject is a cancer patient needing treatment by two or more cancer agents in a synergistic mode; and wherein the cancer is a cancer of the bladder (including accelerated and metastatic bladder cancer), breast (e.g., estrogen receptor positive breast cancer, estrogen, receptor negative breast cancer; HER-2 positive breast cancer; HER-2 negative breast cancer, progesterone receptor positive breast cancer, progesterone receptor negative breast cancer; estrogen receptor negative, HER-2 negative and progesterone receptor negative breast cancer (i.e., triple negative breast cancer); inflammatory breast cancer), colon (including colorectal cancer), kidney (e.g., transitional cell carcinoma), liver, lung (including small and non-small cell lung cancer, lung adenocarcinoma and squamous cell cancer), genitourinary tract, e.g., ovary (including fallopian tube and peritoneal cancers), cervix, prostate, testes, kidney, and ureter, lymphatic system, rectum, larynx, pancreas (including exocrine pancreatic carcinoma), esophagus, stomach, gall bladder, thyroid, skin (including squamous cell carcinoma), brain (including glioblastoma multiforme), head and neck (e.g., occult primary), and Soft tissue (e.g., Kaposi's sarcoma (e.g., AIDS related Kaposi's sarcoma), leiomyosarcoma, angiosarcoma, and histiocytoma), multiple myeloma, and chronic myeloid leukemia.

15. A method of preparing liposomes loaded with one or more active pharmaceutical ingredients, comprising the steps of:
a) preparing a lipid dispersion in a solution comprising a trapping agent(s) and optionally a buffering agent(s) to form a suspension comprising liposome particles;
b) reducing liposome particle size by heating the suspension to an elevated temperature (at or above 50° C.);
c) substantially removing the trapping agent in the suspension outside of the liposomes, thereby obtaining unloaded liposomes;
d) dissolving one or more active pharmaceutical ingredient(s) (API) in an aqueous solution in the presence of a solubility improving agent to obtain an API solution;
e) incubating the unloaded liposomes of step c) with the API solution of step d) comprising the solubility improving agent at an elevated temperature (at or above 50° C.), thereby forming liposome particles comprising an aqueous interior core loaded with the one or more API(s) encapsulated by a bilayer membrane of the lipid, wherein the drug-loaded liposome particles are suspended in an external liquid medium;
f) optionally further removing unloaded drug molecules and the solubility improving agent outside of the liposome particles obtained in step e) by dialysis,
ultracentrifugation, or/and size exclusion chromatography; and
g) optionally forming dry liposome particulates loaded with the one or more API(s) by lyophilizing the liposome particles obtained in step e) or step f).

16. The method of claim 15, wherein the solubility improving agent is selected from cyclodextrins and derivatives (e.g., sulfobutylether-β-cyclodextrin and hydroxypropyl-β-cyclodextrin), polyvinylpyrrolidone, polyethylene glycol and derivatives, sorbitol, non-ionic surfactants, and salts thereof, or a combination thereof.

17. The method of claim 15, wherein at the beginning of drug loading step e), the liquid medium of the liposome interior core has a pH in the range from about 5.0 to about 10.0 and the exterior medium outside of the liposome particles has a pH in the range from about 2.0 to about 5.0; and wherein the step e) of incubating the unloaded liposomes with the drug solution results in at least 50% of total API(s) being encapsulated within the aqueous interior core of the liposome particles and less than 50% of total API molecules existing in the external liquid medium.

18. A treatment kit, comprising a first container comprising a plurality of the drug-loaded liposome particles according to claim 1, and a second container comprising a liposome dispersion liquid medium, wherein the drug-loaded liposome particles and the liposome dispersion liquid medium can be mixed in either the first container or the second container to form a dispersion that is ready for administration to a subject in need of treatment.

19. A treatment kit, comprising a container comprising a liposome pharmaceutical composition according to claim 10 ready for administration to a subject in need of treatment.

20. A method of treating a cancer in a subject in need of treatment, the method comprising administering the subject a therapeutically effective amount of the pharmaceutical composition of claim 10, wherein the cancer is selected from bladder cancer, breast cancer, colon cancer, kidney cancer, liver cancer, lung cancer, genitourinary tract cancer, ovary cancer, cervix cancer, prostate cancer, testes cancer, kidney cancer, ureter cancer, lymphatic system cancer, rectum cancer, larynx cancer, pancreatic cancer, esophagus cancer, stomach cancer, gall bladder cancer, thyroid cancer, skin cancer, brain cancer, head and neck cancer, soft tissue cancer, multiple myeloma, and chronic myeloid leukemia.
